(19) United States
(12) Patent Application Publication
(10) Pub. No.: US 2024/0188880 A1
(43) Pub. Date: Jun. 13, 2024

(54) INFORMATION PROCESSING APPARATUS, INFORMATION PROCESSING SYSTEM, AND INFORMATION PROCESSING METHOD

(71) Applicant: PARAMOUNT BED CO., LTD., Tokyo (JP)

(72) Inventor: Yasuhiro KASAHARA, Tokyo (JP)

(73) Assignee: PARAMOUNT BED CO., LTD., Tokyo (JP)

(21) Appl. No.: 18/533,824

(22) Filed: Dec. 8, 2023

(30) Foreign Application Priority Data
Dec. 8, 2022 (JP) 2022-196412

(51) Int. Cl. A61B 5/00; A61B 5/11; A61B 5/16; A61B 10/00

(52) U.S. Cl. CPC A61B 5/4812; A61B 5/0002; A61B 5/11; A61B 5/165; A61B 5/6891; A61B 5/7246; A61B 10/00; A61B 2010/0003; A61B 2505/07

(57) ABSTRACT

An information processing apparatus includes: an acquisition unit configured to acquire, based on an output from a detection device that can detect a body movement of a user who receives care assistance, body movement information including information related to a sleep state of the user; a storing unit configured to store information in which a response content performed by a skilled worker to the user has been associated with first body movement information serving as the time series body movement information; and a processing unit configured to perform processing of presenting, when having acquired a request including second body movement information serving as the time series body movement information, similar information including a similarity between the second body movement information and the first body movement information, and the response content associated with the first body movement information.

### CROSS-REFERENCE TO RELATED APPLICATIONS

This Non-provisional application claims priority under 35 U. S. C. § 119 (a) on Patent Application No. 2022-196412 filed in Japan on Dec. 8, 2022, the entire contents of which are hereby incorporated by reference.

### FIELD

The present application relates to an information processing apparatus, an information processing system, an information processing method, and the like.

### BACKGROUND

Systems that manage health states and the like of users have been known. For example, JP2019-503017A discloses a system and the like that share real-time measurement data on a user (patient).

### BRIEF DESCRIPTION OF THE DRAWINGS

FIG. 1 illustrates an example of an information processing system including an information processing apparatus.

FIG. 2 illustrates a configuration example of a server system.

FIG. 3 illustrates a configuration example of a terminal device.

FIG. 4 illustrates an example of a detection device serving as one example of a measuring device.

FIG. 5 illustrates a detailed example of the information processing system.

FIG. 6 is a diagram illustrating a flow of data in processing in an embodiment.

FIG. 7 is a flowchart illustrating accumulation processing of data.

FIG. 8 is a flowchart illustrating measurement processing of body movement information.

FIG. 9 illustrates an example of a screen to be displayed on a terminal device of a skilled worker.

FIG. 10 illustrates an example of a screen to be displayed on the terminal device of the skilled worker.

FIG. 11 is a diagram illustrating a specific example of body movement information, and an example of execution timing of an input operation.

FIG. 12 is a flowchart illustrating use processing of data.

FIG. 13 illustrates an example of a screen to be displayed on a terminal device of an unskilled worker.

FIG. 14 is a flowchart illustrating similarity calculation processing.

FIG. 15 is a diagram illustrating an example of similarity calculation processing based on a distance.

FIG. 16 illustrates a display screen example of similar information to be displayed on the terminal device of the unskilled worker.

FIG. 17 is another flowchart illustrating the similarity calculation processing.

FIG. 18A is a diagram illustrating a relation example between response contents and comparison periods.

FIG. 18B is a diagram illustrating a relation example between response contents and comparison periods.

FIG. 19A is a diagram illustrating calculation processing of a similarity based on environment information (temperature).

FIG. 19B is a diagram illustrating calculation processing of a similarity based on environment information (humidity).

FIG. 20 illustrates a display screen example of similar information to be displayed on the terminal device of the unskilled worker.

FIG. 21 is a diagram illustrating processing in which emotion information is used.

FIG. 22 is a diagram illustrating different execution timing of the input operation.

FIG. 23A is a diagram illustrating correction processing of a reference point.

FIG. 23B is a diagram illustrating the correction processing of the reference point.

FIG. 24A is another diagram illustrating the correction processing of the reference point.

FIG. 24B is another diagram illustrating the correction processing of the reference point.

FIG. 24C is another diagram illustrating the correction processing of the reference point.

FIG. 25 illustrates a detailed example of the information processing system.

FIG. 26 illustrates an example of a screen to be displayed on a terminal device of a skilled health care worker.

FIG. 27 illustrates an example of a screen to be displayed on a terminal device of an unskilled health care worker.

FIG. 28 illustrates an example of a screen of similar information to be displayed on the terminal device of the unskilled health care worker.

FIG. 29A illustrates an example of a screen to be displayed on a terminal device of a facility manager.

FIG. 29B illustrates an example of a screen to be displayed on the terminal device of the facility manager.

FIG. 30 is a diagram illustrating work evaluation processing based on execution timing of an input operation.

FIG. 31A illustrates an example of a screen to be displayed on the terminal device of the facility manager.

FIG. 31B illustrates an example of a screen to be displayed on the terminal device of the facility manager.

FIG. 32 is a flowchart illustrating database update processing.

FIG. 33 illustrates an example of a screen to be displayed on a terminal device of a family of a care receiver.

### DETAIL DESCRIPTION

One or more embodiments are now described with reference to the drawings, wherein like reference numerals are used to refer to like elements throughout. In the following description, for purposes of explanation, numerous specific details are set forth in order to provide a thorough understanding of the various embodiments. It is evident, however, that the various embodiments can be practiced without these specific details (and without applying particular networked environment or standard).

As used in this disclosure, in some embodiments, the terms “component,” “system” and the like are intended to refer to, or comprise, a computer-related entity or an entity related to an operational apparatus with one or more specific functionalities, wherein the entity can be either hardware, or a combination of hardware and software in execution.

One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software application or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software stored on a non-transitory electronic memory or firmware that confers at least in part the functionality of the electronic components. While various components have been illustrated as separate components, it will be appreciated that multiple components can be implemented as a single component, or a single component can be implemented as multiple components, without departing from example embodiments. Further, the various embodiments can be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer-readable (or machine-readable) device or computer-readable (or machine-readable) storage/communications media having a computer program stored thereon. For example, computer readable storage media can comprise, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

In addition, the words “example” and “exemplary” are used herein to mean serving as an instance or illustration. Any embodiment or design described herein as “example” or “exemplary” is not necessarily to be construed as preferred or advantageous over other embodiments or designs. Rather, use of the word example or exemplary is intended to present concepts in a concrete fashion. As used in this application, the term “or” is intended to mean an inclusive “or” rather than an exclusive “or”. That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Embodiments described herein can be exploited in substantially any wireless communication technology, comprising, but not limited to, wireless fidelity (Wi-Fi), global system for mobile communications (GSM), universal mobile telecommunications system (UMTS), worldwide interoperability for microwave access (WiMAX), enhanced general packet radio service (enhanced GPRS), third generation partnership project (3GPP) long term evolution (LTE), third generation partnership project 2 (3GPP2) ultra mobile broadband (UMB), high speed packet access (HSPA), Z-Wave, Zigbee and other 802.XX wireless technologies and/or legacy telecommunication technologies.

In general, one aspect of the present application is an information processing apparatus including:
an acquisition unit configured to acquire, based on an output from a detection device that can detect a body movement of a user who receives care assistance, body movement information including information related to a sleep state of the user;
a storing unit configured to store information in which a response content performed by a skilled worker to the user has been associated with first body movement information serving as the time series body movement information; and
a processing unit configured to perform processing of presenting, when having acquired a request including second body movement information serving as the time series body movement information, similar information including a similarity between the second body movement information and the first body movement information, and the response content associated with the first body movement information.

Another aspect of the present application is an information processing system including:
a detection device that can detect a body movement of a user who receives care assistance;
a server system configured to acquire body movement information including information related to a sleep state of the user, based on an output from the detection device, and stores information in which a response content performed by a skilled worker to the user has been associated with first body movement information serving as the time series body movement information; and
a terminal device configured to transmit a request including second body movement information serving as the time series body movement information to the server system, in which
the server system is configured to:
perform processing of presenting, when having acquired the request from the terminal device, similar information including a similarity between the second body movement information and the first body movement information, and the response content associated with the first body movement information.

Further another aspect of the present application is an information processing method including:
acquiring, based on an output from a detection device that can detect a body movement of a user who receives care assistance, body movement information including information related to a sleep state of the user;
storing information in which a response content performed by a skilled worker to the user has been associated with first body movement information serving as the time series body movement information; and
performing processing of presenting, when the request including second body movement information serving as the time series body movement information has been acquired, similar information including a similarity between the second body movement information and the first body movement information, and the response content associated with the first body movement information.

Hereinafter, the present embodiments will be described with reference to the drawings. As for the drawings, the same reference numerals are given to the same or similar elements, and redundant descriptions are omitted. Note that, the present embodiments to be described below do not unduly limit the scope of the claims. Moreover, all the configurations to be described in the present embodiments do not need to be essential features in the disclosure.

### 1. First Embodiment

### 1.1 System Configuration Example

An information processing system 10 according to an embodiment provides, for example, in the scene of care assistance (including care), information on a response and the like performed by a care giver for a care receiver, to another care giver. The care giver includes a care manager, a care worker, a home-care worker, and the like. The care giver may include an occupational therapist and a physiotherapist. Specifically, as for work that is performed in accordance with “intuitions” and “tacit knowledge” of a skilled care giver, for example, in scenes of care assistance in a care facility, home-visit care, and the like, the information processing system 10 according to the embodiment may support, by digitalizing the “intuition” and the “tacit knowledge”, the other care givers so as to perform suitable care independent of the degree of proficiency. Hereinafter, the information processing system 10, and an information processing apparatus in the information processing system 10 will be described in detail.

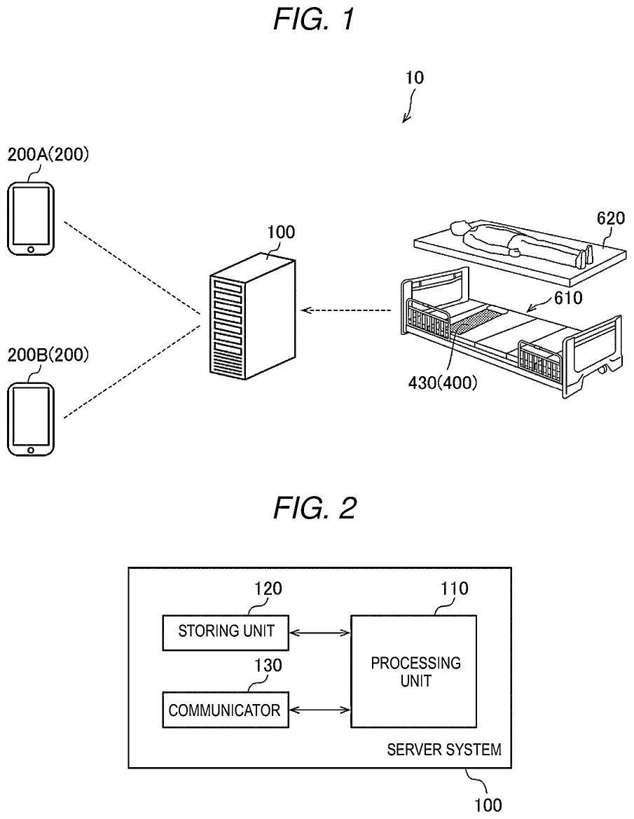

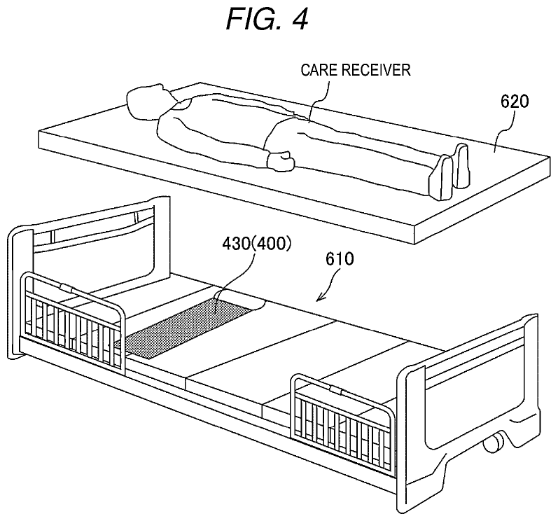

FIG. 1 illustrates a configuration example of the information processing system 10 including the information processing apparatus according to the embodiment. The information processing system 10 includes, for example, a server system 100, a terminal device 200, and a measuring device 400. The server system 100 implements the information processing apparatus, for example. The terminal device 200 may include, for example, a terminal device 200A and a terminal device 200B. The measuring device 400 may be, for example, a detection device 430, which is described later using FIG. 4. The configuration of the information processing system 10 is not limited to that in FIG. 1, but various modifications such as omitting a part of the configuration, adding another configuration, and the like can be made.

The terminal device 200 in FIG. 1 is a device that performs communication with the server system 100, and is a terminal that is used by a care giver, for example. The terminal device 200 is, for example, a mobile terminal device such as a smartphone or a tablet terminal. Note that, the terminal device 200 may be another device including a personal computer (PC), a headset, a wearable device such as augmented reality (AR) glasses and mixed reality (MR) glasses, and the like. A plurality of care givers are assumed in the embodiment, and the terminal device 200 may include a plurality of devices that are used by the different care givers. For example, a first care giver uses the terminal device 200A, and a second care giver different from the first care giver uses the terminal device 200B. In the following description, if a plurality of terminal devices do not need to be distinguished from each other, the terminal devices are simply expressed as the terminal device 200. The terminal device 200 only needs to be a device that accumulates data in the server system 100 or uses the data accumulated in the server system 100, and a user of the terminal device 200 is not limited to a care giver. For example, as is described later in a second embodiment, the terminal device 200 may be a device that is used by a health care worker, a facility manager, a data administrator, and the like.

The measuring device 400 is a device that is disposed in the surrounding of a care receiver, and performs a measurement (sensing) related to the care receiver himself/herself, or an environment of the care receiver. For example, the measuring device 400 may be the detection device 430 that detects sleep information and biological information based on a body movement of the care receiver. Details of the detection device 430 will be described later using FIG. 4.

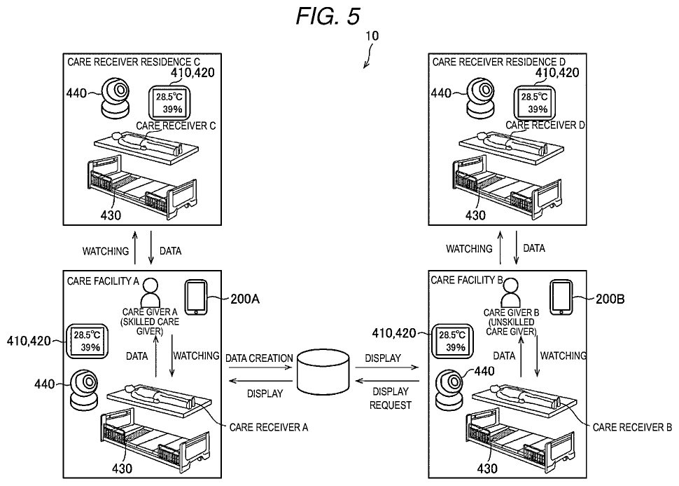

The measuring device 400 is not limited thereto, but may include various devices. For example, the measuring device 400 may be a watch type wearable device that detects sleep information and biological information. The measuring device 400 may include, as is described later using FIG. 5, a thermometer 410, a hygrometer 420, an imaging device 440, and the like. The thermometer 410 detects temperature in the surrounding of a care receiver. The hygrometer 420 detects humidity in the surrounding of a care receiver. The imaging device 440 images a part or all of a care receiver. The measuring device 400 may include a clinical thermometer, an electrocardiograph, a pulse meter, a sphygmomanometer, a pulse oximeter, a body composition meter, a stethoscope, and the like. The measuring device 400 may be a device that automatically operates, or a device that is used by a health care worker such as a nurse.

The server system 100 is connected to the terminal device 200A, the terminal device 200B, and the measuring device 400, via a network, for example. The network herein is, for example, a public communication network such as the Internet. The network is not limited to the public communication network, but may be a local area network (LAN) and the like. The server system 100 may perform communication in conformity with the standard of IEEE802.11, for example. Various modifications for the communication method between the respective devices can be made. For example, although FIG. 1 illustrates an example in which the server system 100 and the measuring device 400 are connected to each other, the measuring device 400 may be connected to another device such as the terminal device 200. In this case, a measurement result in the measuring device 400 is transmitted to the server system 100 via the terminal device 200.

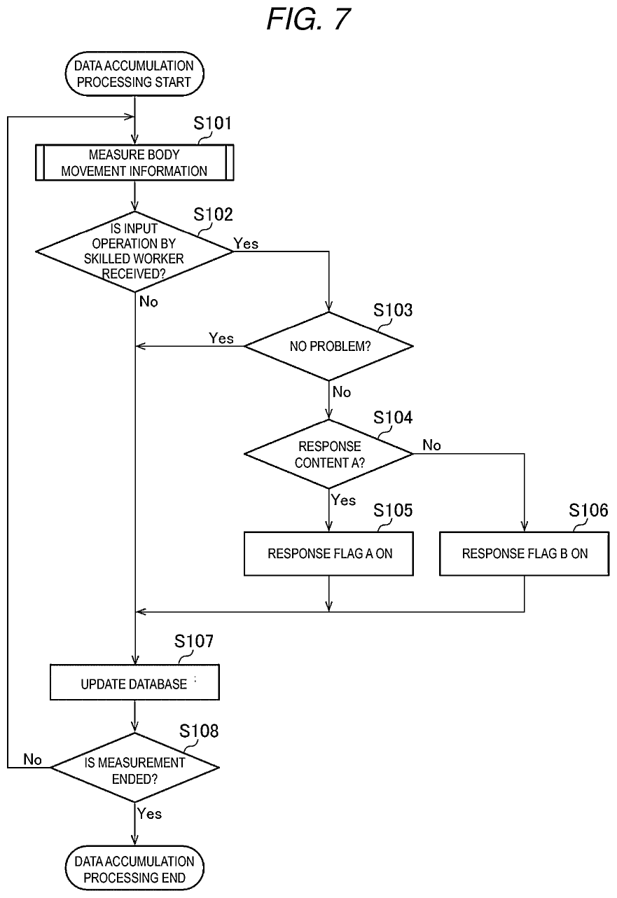

The server system 100 may be one server, or may include a plurality of servers. The server system 100 may include a database server and an application server, for example. The database server may store information that is transmitted from the terminal device 200 and the measuring device 400. The application server performs various kinds of processing based on the information. The application server executes processing related to the server system 100 in FIG. 7 and the like, which is described later, for example. In the following description, the application server may execute at least part of the processing to be executed by the terminal device 200 and the measuring device 400. The plurality of the servers herein may be physical servers or may be virtual servers. In a case where a virtual server is used, the virtual server may be provided in one physical server, or may be disposed in a distributed manner to a plurality of physical servers. As in the foregoing, various kinds of modifications for the specific configuration of the server system 100 in the embodiment can be made.

In a method in the embodiment, the server system 100 collects information such as a measurement result related to a care receiver, and an operation result of the terminal device 200 by a care giver, and performs processing based on the information. For example, the server system 100 may store a measurement result when a skilled care giver has found an abnormality of a care receiver in association with a response content relative to the abnormality performed by the care giver. The server system 100 may provide the stored information to the terminal device 200 of another care giver. This provides the determination and the response by the skilled care giver, and it is possible to prompt a care giver with a low degree of proficiency to perform a suitable response. Hereinafter, a care giver with a low degree of proficiency is also referred to as an unskilled care giver. In at-home care, a family or the like (family care giver) of a care receiver provides care assistance of the care receiver in some cases. A care giver in the embodiment may include a family care giver. In this case as well, it is possible to prompt a family care giver who is not an expert of the care assistance to perform a suitable response.

FIG. 2 is a block diagram illustrating a detailed configuration example of the server system 100. The server system 100 includes, for example, a processing unit 110, a storing unit 120, and a communicator 130. The configuration of the server system 100 is not limited to that in FIG. 2, but modifications such as omitting a part of the configuration, adding another configuration, and the like can be made.

The processing unit 110 in the embodiment is implemented by hardware described below. The hardware can include at least one of a circuit for processing digital signals and a circuit for processing analog signals. For example, the hardware may be implemented by one or a plurality of circuit devices mounted to a circuit substrate and/or one or a plurality of circuit elements. One or a plurality of circuit devices are, for example, an integrated circuit (IC) and a field-programmable gate array (FPGA). One or a plurality of circuit elements are, for example, a resistance and a capacitor.

Moreover, the processing unit 110 may be implemented by processors described below. The server system 100 in the embodiment includes a memory that stores information, and a processor that operates based on the information stored in the memory. The information is, for example, a program and various kinds of data. The memory may be the storing unit 120, or may be another memory. The processor includes hardware. As the processors, various kinds of processors including a central processing unit (CPU), a graphics processing unit (GPU), a digital signal processor (DSP), and the like can be used. The memory may be a semiconductor memory such as a static random access memory (SRAM), a dynamic random access memory (DRAM), and a flash memory, may be a register, may be a magnetic storage device such as a hard disk device (HDD: hard disk drive), and may be an optical storage device such as an optical disc device. For example, the memory stores an instruction readable by a computer, and the processor executes the instruction, thereby implementing a function of the processing unit 110 as processing. The instruction herein may be an instruction in an instruction set constituting the program, or may be an instruction to instruct the hardware circuit of the processor to operate.

The storing unit 120 is a work area of the processing unit 110, and stores various kinds of information. The storing unit 120 can be implemented by various kinds of memories, and the memory may be a semiconductor memory such as SRAM, DRAM, a read only memory (ROM), and a flash memory, may be a register, may be a magnetic storage device, and may be an optical storage device.

The communicator 130 is an interface for performing communication via the network, and includes, in a case where the server system 100 performs wireless communication, for example, an antenna, a radio frequency (RF) circuit, and a base band circuit. The server system 100 may perform wired communication, and the communicator 130 in that case may include a communication interface such as an Ethernet connector, a control circuit of the communication interface, and the like. The communicator 130 may operate in accordance with the control by the processing unit 110, or may include a processor for communication control different from the processing unit 110. The communicator 130 may perform communication in accordance with a scheme defined in IEEE802.11 and IEEE802.3, for example. Various kinds of modifications for a specific communication scheme can be made.

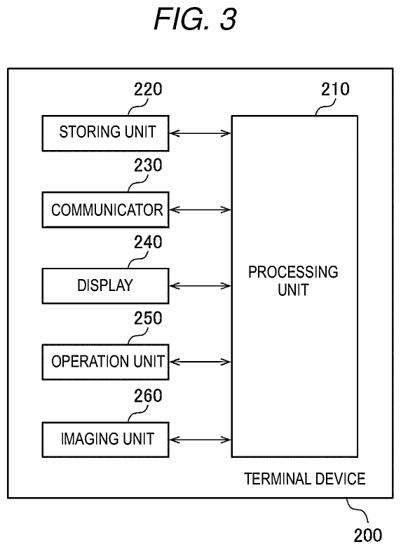

FIG. 3 is a block diagram illustrating a detailed configuration example of the terminal device 200. The terminal device 200 may include, for example, a processing unit 210, a storing unit 220, a communicator 230, a display 240, an operation unit 250, and an imaging unit 260. The configuration of the terminal device 200 is not limited to that in FIG. 3, but modifications such as omitting a part of the configuration, adding another configuration, and the like can be made.

The processing unit 210 is implemented by hardware including at least one of a circuit for processing digital signals and a circuit for processing analog signals. The processing unit 210 may be implemented by processors. As the processors, various kinds of processors including CPU, GPU, DSP, and the like can be used. The processor executes an instruction stored in the memory of the terminal device 200, thereby implementing the function of the processing unit 210 as processing.

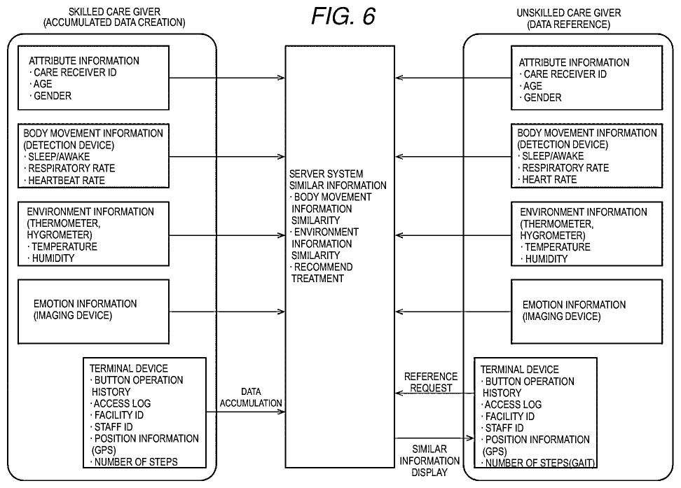

The storing unit 220 is a work area of the processing unit 210, and is implemented by various kinds of memories including SRAM, DRAM, ROM, and the like. The storing unit 220 may store a history and the like of an input operation by a care giver. The storing unit 220 may store various kinds of information related to a care giver who uses the terminal device 200. A specific example of the information is described later using FIG. 6.

The communicator 230 is an interface for performing communication via the network, and includes, for example, an antenna, an RF circuit, and a base band circuit. The communicator 230 performs communication with the server system 100 via the network, for example. The communicator 230 may execute wireless communication in conformity with the standard of IEEE802.11, for example, with the server system 100.

The display 240 is an interface that displays various kinds of information, and may be a liquid crystal display, may be an organic EL display, or may be a display using another scheme. The operation unit 250 is an interface that receives a user operation. The operation unit 250 may be a button or the like that is provided in the terminal device 200. Moreover, the display 240 and the operation unit 250 may be integrally configured as a touch panel.

The imaging unit 260 includes an image sensor that outputs image information by imaging a predetermined imaging range. The image information herein may be a still image or may be a moving image. Image information may be color or may be monochrome. The imaging unit may include a depth sensor that detects a distance to a photographed subject, or may include a sensor (for example, an infrared sensor) or the like that detects heat of the photographed subject.

The terminal device 200 may include a configuration, which is not illustrated in FIG. 3. For example, the terminal device 200 may include various kinds of sensors including a motion sensor such as an acceleration sensor and a gyroscope sensor, a a pressure sensor, a global positioning system (GPS) sensor, and the like. Moreover, the terminal device 200 may include a light emission unit, a vibration unit, a sound input unit, a sound output unit, and the like. The light emission unit is, for example, a light emitting diode (LED), and makes a notification by light emission. The vibration unit is, for example, a motor, and makes a notification by vibration. The sound input unit is a microphone, for example. The sound output unit is, for example, a speaker, and makes a notification by sound. The plurality of the terminal devices 200 are not limited to each having the same configuration. For example, the terminal device 200A and the terminal device 200B may be devices having different configurations.

FIG. 4 is a diagram illustrating an example of the measuring device 400, and an example of the detection device 430 that is disposed in sections of a bed 610. The detection device 430 is a sheet-shaped or plate-shaped device that is provided between the sections of the bed 610 and a mattress 620, for example, as illustrated in FIG. 4.

The detection device 430 includes a pressure sensor (for example, pneumatic sensor) that outputs a pressure value, and is a device that detects a body vibration (body movement, vibration) of a user via the mattress 620 when the user has gone to bed. Hereinafter, information indicating body vibration that is detected by a pressure sensor of the detection device 430 is expressed as body movement information. The body movement information in the embodiment is not limited to information (body movement information in the narrow sense) indicating the body vibration, but may include various kinds of information to be obtained based on the information.

For example, the body movement information may include sleep information that is obtained based on the body vibration (body movement information in the narrow sense) detected by the detection device 430. The sleep information herein includes various kinds of information that is obtained by analysis processing of the body movement information, for example, information related to breathing, heartbeat, sleep or awake, an amount of activity, a posture, and bed leaving/bed presence. The sleep information is not limited to information related to a user who is sleeping, but may include information related to a user who is awaken.

The information indicating breathing may be the number of times of breathing (hereinafter, expressed as a respiratory rate) per unit time. The information related to heartbeat may be the number of times of beating (hereinafter, expressed as a heart rate) per unit time. The unit time herein is, for example, one minute, but different time may be used. For example, the detection device 430 may calculate a respiratory rate and a heart rate from a peak frequency by analyzing the periodicity of the body movement. The analysis of the periodicity is a Fourier transform, for example.

Information indicating the breathing is not limited to the respiratory rate. For example, PCT/JP2010/063892 “DEVICE FOR CALCULATING RESPIRATORY WAVEFORM INFORMATION AND MEDICAL DEVICE USING RESPIRATORY WAVEFORM INFORMATION” filed on Aug. 11, 2010 describes a device that obtains an indicator indicating the degree of f stability of breathing based on the periodicity or the fluctuation of the breathing, for example. This patent application is fully incorporated in the specification of the present application by reference. For example, information indicating breathing in the embodiment may be the periodicity or the fluctuation of the breathing, or may be an indicator indicating the degree of stability. Information indicating the heart rate may be similarly the periodicity or the fluctuation of the heart rate, or information indicating the degree of stability.

It is known that values of the respiratory rate and the heart rate in a sleep state are lower than those in an awake state. Determination of sleep or awake may be made based on the abovementioned respiratory rate or heart rate. When information indicating sleep or awake is obtained, information related to an amount of activity or a posture, which is described later, may be used. For example, the sleep state is determined in a case where the amount of activity equal to or less than a predetermined amount, or in a case where the frequency of posture change is equal to or less than a predetermined frequency. The sleep state may be subdivided into non-REM sleep and REM sleep, and a depth of the sleep may be obtained.

The detection device 430 detects body vibration per sampling unit time, and may output the number of times of the body vibration as an amount of activity. It is grasped that the body weight is applied to which position of the bed 610 based on the distribution of pressure values, so that the detection device 430 can estimate a posture of a care receiver. A pressure value to be detected decreases in the bed leaving state of the user compared with in the bed presence, so that the detection device 430 can make a determination of bed leaving or bed presence based on the pressure value and a time-series change in the pressure value.

The detection device 430 may perform processing of obtaining sleep information including a respiratory rate and the like based on the pressure value. For example, the detection device 430 may include a memory that stores a pressure value, and a processor that performs the abovementioned processing based on the pressure value. The method in the embodiment is not limited thereto, but another device may calculate a respiratory rate and the like. For example, the server system 100 or the terminal device 200 may acquire data indicating body vibration output from a pressure sensor, and perform the abovementioned processing, thereby obtaining sleep information including a respiratory rate and the like.

FIG. 5 is a diagram illustrating an example of the information processing system 10 in the embodiment, and a more specific use case. For example, devices in the information processing system 10 may be distributed and disposed so as to support care assistance in a plurality of places. In the example of FIG. 5, care receivers respectively receive care assistance in four locations of a care facility A, a care facility B, a care receiver residence C, and a care receiver residence D. At least a care receiver A occupies the care facility A where the detection device 430, thermometer 410, the hygrometer 420, and the imaging device 440 are disposed as the measuring devices 400 that perform measurements related to the care receiver A. The same applies to the care facility B, the care receiver residence C, and the care receiver residence D, in each of which the detection device 430, the thermometer 410, the hygrometer 420, and the imaging device 440 for providing care assistance to a care receiver B, a care receiver C, or a care receiver D are disposed. The number and the type of the measuring devices 400 to be disposed may be different for every care receiver.

Care givers respectively work at the care facility A and the care facility B. In the example of FIG. 5, a care giver A with a high degree of proficiency works at the care facility A, and a care giver B with a low degree of proficiency works at the care facility B.

The care giver A provides care assistance to the care receiver A who occupies the care facility A. For example, the care giver A carries the terminal device 200A, and a measurement result of the measuring device 400 disposed in the surrounding of the care receiver A is displayed in the terminal device 200A. The care giver A determines whether an abnormality is present in the care receiver A based on the measurement result, and performs some response when having found the abnormality.

The care giver A may provide care assistance to the care receiver C who receives at-home care (remote care assistance) in the care receiver residence C. For example, the measuring device 400 disposed in the care receiver residence C transmits a measurement result to the terminal device 200A directly or via another device. For example, the measuring device 400 may transmit a measurement result to the terminal device 200A via the server system 100 or a management PC that is disposed in the care facility A. The care giver A determines whether an abnormality is present in the care receiver C based on the measurement result, and performs some response when having found the abnormality. In this case, since the care receiver C and the care giver A are present in places away from each other, responses to be executed by the care giver A may include responses specific to at-home care, such as a visit to the care receiver residence, an online consultation, and the like.

The same applies to the care giver B. The care giver B carries the terminal device 200B, and a measurement result related to the care receiver B who occupies the care facility B and a measurement result related to the care receiver D who receives at-home care in the care receiver residence D are displayed in the terminal device 200B. The care giver B executes care assistance to the care receiver B and remote care assistance to the care receiver D, based on the measurement results.

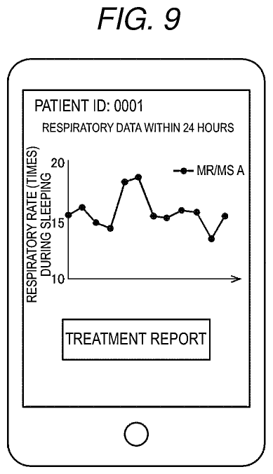

In this way, data including a measurement result related to a care receiver is collected in the terminal device 200 to be used by a care giver, and watching of the care receiver is executed based on the data. A display example of a measurement result in the terminal device 200 will be described later using FIG. 9 and the like.

However, even if a care giver with a low degree of proficiency browses the measurement result by the measuring device 400, there is a possibility that the care giver is difficult to suitably respond. For example, if a care giver with a low degree of proficiency has known the presence of an abnormality in the sleep information, the respiratory rate, or the like, the care giver cannot determine a response suitable to the abnormality. In the example of FIG. 5, when the care giver B with a low degree of proficiency has browsed a measurement result related to the care receiver B or the care receiver D, there is a possibility that the care giver B cannot suitably determine a response.

The information processing apparatus in the embodiment may include an acquisition unit, a storing unit, and a processing unit. The acquisition unit acquires, based on an output from the detection device 430 that detects a body movement of a user who receives care (care assistance), body movement information including information related to a sleep state of the user. The user herein is specifically a care receiver who receives care assistance service. As is described later in the second embodiment, the user in the embodiment may be a patient who receives medical service. Hereinafter, although an example in which a user who receives at-home care is a care receiver is described in the first embodiment, a “care receiver” in the present specification can be replaced with a “patient” as appropriate.

The storing unit stores information in which a response content performed by a skilled worker to a user (care receiver) has been associated with first body movement information serving as time-series body movement information. The processing unit performs, in a case where the processing unit has acquired a request including second body movement information serving as time-series body movement information, processing of presenting similar information including a similarity between the second body movement information and the first body movement information. The similar information includes, for example, as is described later using FIG. 16, a numerical value indicating the similarity of the body movement information, and a response content associated with the first body movement information. The presenting processing of similar information is not limited to presenting processing of only presenting the numerical value and the response content. For example, in a case where a plurality of first body movement information to be compared are present, the processing unit may sort and present the plurality of the first body movement information (and the associated response contents) based on the similarity. Alternatively, the processing unit may perform processing of only selecting first body movement information having a similarity equal to or higher than a predetermined similarity as a presentation target. The processing unit may perform processing of changing a presenting mode in accordance with a value of the similarity, a type of the response content, and the like. The change in the presenting mode herein may be changes in colors of a character and a background that are used when the display processing is performed, for example, or changes in a size and a type of a font, or may be another processing.

For example, in a case where the server system 100 implements the information processing apparatus, the acquisition unit may correspond to the communicator 130 in FIG. 2. For example, the communicator 130 receives measurement results of the measuring devices 400 disposed in the surroundings of the respective care receivers, directly or via the terminal device 200A and the terminal device 200B. The measurement result includes body movement information from the detection device 430, and the body movement information includes sleep information. The storing unit of the information processing apparatus may correspond to the storing unit 120 in FIG. 2. In the example of FIG. 5, the skilled worker is the care giver A, and the first body movement information is part of the measurement results by the measuring devices 400 disposed in the surroundings of the care receiver A and the care receiver C. The processing unit of the information processing apparatus corresponds to the processing unit 110 in FIG. 2. As mentioned above, the acquisition unit of the information processing apparatus is not limited to that to acquire body movement information calculated in the detection device 430, but may acquire a pressure value from the detection device 430 and calculate body movement information based on the pressure value. The acquisition unit is not limited to that to correspond to the communicator 130, but may correspond to the processing unit 110 that performs computing processing of body movement information based on the pressure value.

With the method in the embodiment, similar information between body movement information (first body movement information) in a scene in which a skilled worker has performed some response and body movement information (second body movement information) in a different scene is displayed. For example, in a case where a similarity between the first body movement information and the second body movement information is high, it is assumed that situations of the care receivers are similar in these two scenes. The probability that the response associated with the first body movement information is effective for the care receiver whose second body movement information has been acquired is high. In other words, similar information becomes information useful for the determination of a suitable response. For example, in a situation where an unskilled care giver cannot determine a specific response while recognizing an abnormality in body movement information, presenting similar information makes it possible to support the response determination by the care giver. As a result, it is possible to execute a suitable response independent of the degree of proficiency of the care giver.

FIG. 6 is a diagram illustrating a flow of data in the embodiment. As illustrated in FIG. 6, a skilled care giver creates accumulated data for reference. Specifically, the measuring device 400 such as the thermometer 410, the hygrometer 420, the detection device 430, or the imaging device 440 is disposed in the surrounding of a care receiver who is taken charge by a skilled care giver. The detection device 430 transmits body movement information such as information related to sleep or awake, a respiratory rate, and a heart rate, to the server system 100. The thermometer 410 and the hygrometer 420 respectively transmit the temperature and the humidity, to the server system 100. The imaging device 440 transmits emotion information based on an imaged image imaged by a care receiver, to the server system 100. As is described later, the imaging device 440 may transmit an imaged image to the server system 100, and the processing unit 110 may obtain emotion information based on the imaged image.

As illustrated in FIG. 6, the server system 100 may acquire information related to an attribute of a care receiver. The attribute includes an ID that uniquely specifies a care receiver, and age, gender, a body height, a body weight, an anamnesis, a medication history, and the like of the care receiver. The server system 100 may acquire an electronic medical record of the care receiver, which is not illustrated in FIG. 6. Information of the electronic medical record may be a result of blood inspection, for example, or may include information on another item.

The terminal device 200 (for example, the terminal device 200A in FIG. 5) that a skilled care giver uses transmits various kinds of information including an input operation to the server system 100. Specifically, the terminal device 200 transmits an operation history of a button, an access log to the system according to the embodiment or another service, a facility ID for specifying a facility to which a care giver belongs, a staff ID for specifying a care giver, position information based on GPS, information on the number of steps (GAIT, which can be measured by an accelerometer), and the like, to the server system 100. The operation history herein includes a response content executed by the skilled care giver. In a case where care recording software that manages a history, a schedule, and the like of care is used in a care facility, recording contents by the care recording software may be transmitted to the server system 100. The recording contents of the care recording software may be transmitted from a device (for example, a management terminal in the facility) to which the software has been installed to the server system 100, or may be transmitted from the terminal device 200 to the server system 100.

The server system 100 stores the information in the storing unit 120 (database) as accumulated data for reference.

An unskilled worker refers to accumulated data stored in the server system 100. For example, the measuring device 400 such as the thermometer 410, the hygrometer 420, the detection device 430, or the imaging device 440 is disposed in the surrounding of a care receiver who is taken charge by an unskilled care giver. Similar to the example of the skilled care giver, body movement information, a temperature, a humidity, emotion information, and the like are acquired. An attribute of the care receiver and information related to an electronic medical record may be acquired. The unskilled care giver performs a response when an abnormality occurs by browsing these measurement results. In a case where the unskilled care giver cannot determine a suitable response, the terminal device 200 (for example, the terminal device 200B in FIG. 5) transmits a reference request of similar data to the server system 100, based on a user operation.

The server system 100 calculates, based on the request, a similarity between various information acquired based on the measuring device 400 at the skilled worker side and various information acquired based on the measuring device 400 at the unskilled worker side. For example, as mentioned above, a similarity in body movement information is obtained in the embodiment. Similarities in temperature, humidity, and the like may be obtained. In the comparison processing, emotion information, information on an attribute, an electronic medical record, and the like may be referred. The server system 100 performs processing of presenting similar information including the similarity to the terminal device 200 of the unskilled care giver. This can prompt the unskilled care giver to perform a suitable response.

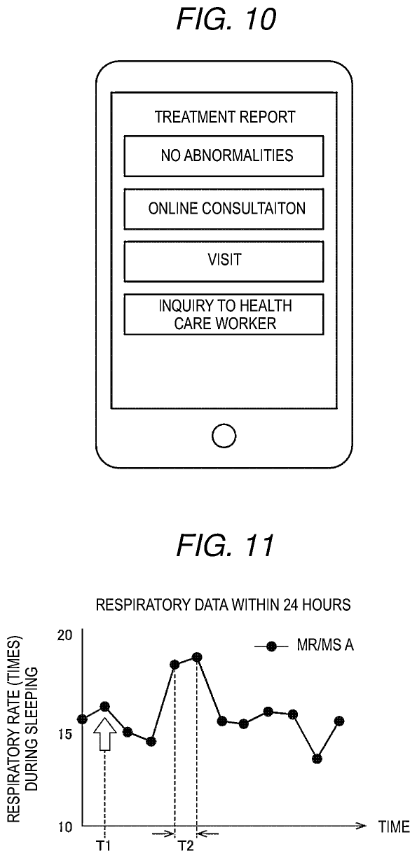

In the method in the embodiment, the abovementioned response content may include the necessity or unnecessity of a visit to a care receiver residence by a care giver. For example, as is described later using FIG. 10, the response content includes “VISIT”, and the necessity or unnecessity of the visit may be presented to a user (unskilled worker in the narrow sense) of the terminal device 200 based on whether the response content is presented. As is a relation between the care giver B and the care receiver D in FIG. 5, in the embodiment, a case where a care receiver receives remote care assistance at a position distant from a care giver can be considered. The accuracy of a response is especially important in the remote care assistance, because the care giver needs to consider a response including whether the care giver visits a distant care receiver. In that regard, information related to the necessity or unnecessity of a visit is accumulated and used in the embodiment, so that it is possible to present information suitable for the remote care assistance. For example, the care giver A with a high degree of proficiency executes a response including the necessity or unnecessity of a visit as a response to the care receiver C who receives at-home care, and a result thereof is accumulated in the server system 100. Similar information is presented based on such data, so that it is possible to prevent the care giver B with a low degree of proficiency from making an unnecessary visit, or postponing a visit in spite of the occurrence of an abnormality with a high degree of seriousness, for example. As a result, it is possible to prevent another work from stagnating due to the expense in time and labor to a visit, or a state of the care receiver from becoming worse, for example.

The information processing apparatus in the embodiment is not limited to the information processing apparatus that is implemented by the server system 100, but may be implemented by distributed processing of a plurality of devices including the server system 100. For example, the information processing apparatus may be implemented by distributed processing of the server system 100 and the terminal device 200.

A method in the embodiment can be applied to the information processing system 10. As illustrated in FIGS. 1 and 5, the information processing system 10 includes the detection device 430, the server system 100, and the terminal device 200. The detection device 430 detects a body movement of a user (care receiver) who receives at-home care. The server system 100 acquires body movement information including information related to a sleep state of the user based on the output from the detection device 430, and stores information in which a response content performed by a skilled worker to the user has been associated with first body movement information serving as time-series body movement information. The terminal device 200 transmits a request including second body movement information serving as time-series movement body information to the server system. The terminal device 200 herein is the terminal device 200B that an unskilled worker uses in the narrow sense, but the terminal device 200A that a skilled worker uses is not prevented from transmitting the abovementioned request. In a case where the server system 100 has acquired the request from the terminal device 200, the server system 100 performs processing of presenting similar information including the similarity between the second body movement information and the first body movement information, and the response content associated with the first body movement information.

Part or all of the processing that is performed by the information processing system 10 in the embodiment may be implemented by a program. The processing that is performed by the information processing system 10 is processing that is performed by the processing unit 110 of the server system 100 in the narrow sense, but may be processing that is performed by the processing unit 210 of the terminal device 200. The processing that is performed by the information processing system 10 may include processing that is executed by a processor in the measuring device 400.

A program according to the embodiment can be stored in a non-temporary information storage medium (information storage device) that is a medium readable by a computer, for example. The information storage medium can be implemented by, for example, an optical disc, a memory card, an HDD, or a semiconductor memory. The semiconductor memory is an ROM, for example. The processing unit 110 and the like perform the various kinds of the processing in the embodiment based on a program that is stored in the information storage medium. In other words, the information storage medium stores the program for causing a computer to function as the processing unit 110 and the like. The computer is an apparatus that is provided with an input device, a processing unit, a storing unit, and an output unit. Specifically, the program according to the embodiment is a program for causing the computer to execute respective steps, which are described later using FIG. 7 and other drawings.

The method in the embodiment can be applied to an information processing method including the following respective steps. An information processing method includes: a step of acquiring, based on an output from a detection device that detects a body movement of a user who receives care assistance, body movement information including information related to a sleep state of the user; a step of storing information in which a response content performed by a skilled worker to the user has been associated with first body movement information serving as the time series body movement information; and a step of performing processing of presenting, when the request including second body movement information serving as the time series body movement information has been acquired, similar information including a similarity between the second body movement information and the first body movement information, and the response content associated with the first body movement information.

### 1.2 Flow of Processing

In the embodiment, accumulation processing of acquiring and accumulating information (first body movement information and a response content) from a skilled worker, and use processing of presenting similar information based on the accumulated information, are performed. Hereinafter, each processing will be described.

### <Accumulation Processing>

FIG. 7 is a flowchart illustrating accumulation processing. When the processing is started, firstly, at Step S101, the detection device 430 performs a measurement of body movement information related to a target care receiver. The detection device 430 transmits body movement information serving as a measurement result to the terminal device 200 of a care giver who is a person in charge of the care receiver.

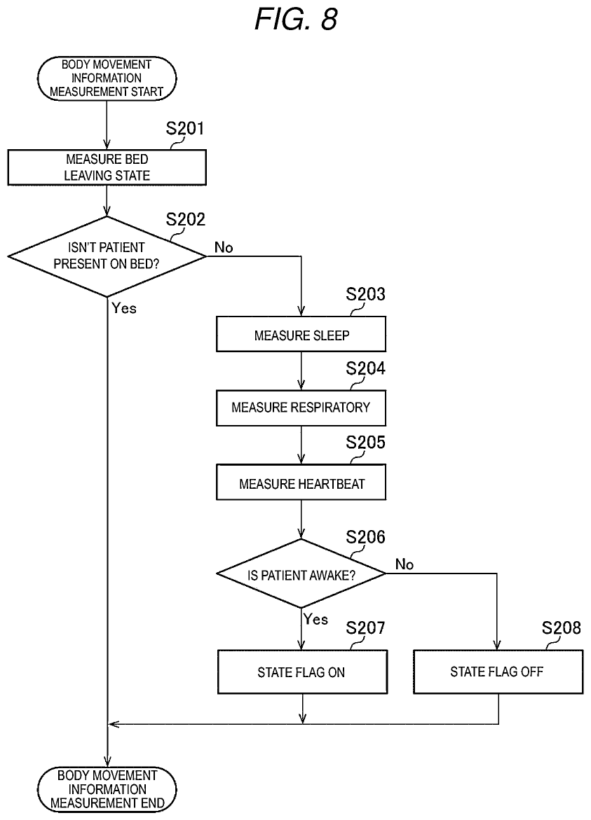

FIG. 8 is a flowchart illustrating the measurement processing of body movement information indicated at Step S101. At Step S201, the detection device 430 obtains a state of bed presence or bed leaving based on a pressure value. For example, the detection device 430 determines a bed presence state in a case where a pressure value is equal to or more than a predetermined value, and determines a bed leaving state in a case where a pressure value is smaller than a threshold.

At Step S202, the detection device 430 determines whether the care receiver is in a bed leaving state based on the information acquired at Step S201. If the bed leaving state has been determined (Step S202: Yes), the detection device 430 cannot detect a body movement of the care receiver, and thus ends the measurement processing of body movement information.

If the bed presence state has been determined (Step S202: No), at Steps S203, S204, and S205, the detection device 430 measures sleep information, a respiratory rate, and a heart rate. The sleep information may be information for identifying either of an awake state and a sleep state, or may include information indicating the depth of sleep. The respiratory rate and the heart rate respectively indicate, for example, the respiratory frequency and the number of times of beating per unit time. The processing of obtaining each information is as mentioned above. The processing at Steps S203-S205 is not limited to the processing that is executed in this order, but may be executed in a different order, or a plurality of processing may be executed in parallel.

At Step S206, the detection device 430 determines whether the care receiver is in an awake state or a sleep state. For example, the detection device 430 may make the determination at Step S206 based on the sleep information acquired at Step S203.

If the care receiver is in an awake state (Step S206: Yes), at Step S207, the detection device 430 sets a state flag to a first bit (for example, 1). The state flag herein is flag information for identifying an awake state or a sleep state of the care receiver, and is included in the body movement information. Herein, an example in which the awake state corresponds to the first bit (1) of the state flag, and the sleep state corresponds to a second bit (for example, 0) of the state flag is illustrated. If the care receiver is in a sleep state (Step S206: No), at Step S208, the detection device 430 sets the state flag to the second bit (for example, 0).

Referring back to FIG. 7, the description is continued. After the body movement information acquired at Step S101 has been transmitted to the terminal device 200 of the care giver, at Steps S102-S106, processing of the terminal device 200 is performed. Firstly, at Step S102, the terminal device 200 executes processing of displaying the body movement information on the display 240, and a determination whether an input operation by the care giver (skilled worker) has been received.

FIG. 9 illustrates an example of a screen to be displayed in the display 240 of the terminal device 200 at Step S102. The display 240 displays an ID for specifying a care receiver to be measured, and displays a measurement result related to the care receiver. FIG. 9 illustrates an example in which as a measurement result, a graph indicating a change in the respiratory rate is displayed. The respiratory rate herein is, for example, a respiratory rate during sleeping, but may include a respiratory rate during awakening. The horizontal axis represents time, and the longitudinal axis represents the respiratory rate per one minute, in the graph. Herein, an example in which old data in a time series manner is displayed at the right side of the graph and new data is displayed at the left side of the graph is illustrated. In other words, the flow of time is a direction heading from the right side of the graph toward the left side. The same applies to a subsequent display example of a measurement result. FIG. 9 illustrates an example in which transition of the respiratory rate for 24 hours is displayed, but a period during which the respiratory rate is to be displayed can be changed. The care giver who uses the terminal device 200 determines whether an abnormality is present in the care receiver by browsing the screen and the like illustrated in FIG. 9. FIG. 9 illustrates the example in which the respiratory rate is displayed, but another body movement information such as sleep information and the heart rate may be displayed. The information may be arranged and displayed on one screen, or may be switchable by receiving a selection operation of a tab, which is not illustrated in FIG. 9.

An object for registering a response to be performed by a care giver when an abnormality occurs to the server system 100 may be displayed in the display 240 of the terminal device 200. The object herein may be a treatment report button illustrated in FIG. 9, for example. For example, in a case where a skilled care giver has determined that an abnormality occurs in a care receiver based on the transition of the body movement information, the skilled care giver executes the selection operation of the treatment report button when starting a response.

FIG. 10 illustrates an example of a screen to be displayed in the display 240 in a case where the selection operation of the treatment report button has been performed. For example, the display 240 may display candidates for a specific response content. FIG. 10 illustrates the example in which four response contents of “NO ABNORMALITIES”, “ONLINE CONSULTAITON”, “VISIT”, and “INQUIRY TO HEALTH CARE WORKER” are displayed as candidates.

“NO ABNORMALITIES” indicates that although a numerical value of body movement information is deviated from that in a usual state, the skilled worker has determined that the state is not serious, and no special response is necessary.

“ONLINE CONSULTAITON” indicates that a care giver remotely checks a state of a care receiver based on an application and the like for performing a Web conference and a video call, for example. For example, a terminal for the care receiver, such as a smartphone or a PC, with the application installed is disposed in the surrounding of the care receiver, and the online consultation is executed by using the terminal device 200 of the care giver and the terminal for the care receiver. The imaging device 440 serving as the measuring device 400 may be used for acquiring an image of the care receiver.

“VISIT” indicates that a care giver visits a care receiver residence, and performs some response face-to-face. “VISIT” may be subdivided so as to include detailed response contents to be executed during the visit. For example, more detailed response contents such as “VISIT: CARE ASSISTANCE OF EXCRETION” and “VISIT: CARE ASSISTANCE OF MEAL” may be selectable.

“INQUIRY TO HEALTH CARE WORKER” indicates that a health care worker who is a medical expert such as a medical doctor or a nurse is asked for a response from a medical standpoint. For example, a care giver who is not a medical expert is difficult to determine whether an abnormality of the respiratory rate or the like is caused by a disease, and execute a medical practice such as collection of blood. In a case where a skilled care giver has determined that a response to the abnormality in the care receiver may be beyond the range of care assistance, the skilled care giver may select “INQUIRY TO HEALTH CARE WORKER”.

If the selection operation of the response content by the skilled worker has been received (Step S102: Yes), the processing unit 210 of the terminal device 200 determines whether a skilled worker has performed some response at Step S103. In a case of the example illustrated in FIG. 10, the processing unit 210 may determine whether the response content selected by the skilled worker is “NO ABNORMALITIES”.

If the skilled worker selects the candidates other than “NO ABNORMALITIES”, and performs some response (Step S103: No), the processing unit 110 performs processing of specifying a specific response content based on the operation input by the skilled worker. For example, in a case where a response content A and a response content B become candidates as a response content, the processing unit 210 may determine whether the skilled worker has selected the response content A at Step S104. If the skilled worker has selected the response content A (Step S104: Yes), the processing unit 210 sets a response flag A to the first bit (1) at Step S105. If the skilled worker has selected the response content B (Step S104: No), the processing unit 210 sets a response flag B to the first bit (1) at Step S106. The response flag A is flag data that is set to the first bit in a case where the response content A has been conducted, and the response flag B is flag data that is set to the first bit in a case where the response content B has been conducted. As mentioned above, the response content may have three or more candidates, and flag data indicating the response content is similarly set in this case as well. For example, as processing corresponding to Steps S104-S106, the processing unit 210 may set a value of flag data capable of specifying which response content among three or more candidates has been selected (for example, three or more flag data, or flag data including a plurality of bits). The processing unit 210 performs processing of associating the set flag data with the body movement information acquired at Step S101.

The terminal device 200 transmits the body movement information with the response content (flag data) associated to the server system 100 via the communicator 230. At Step S107, the processing unit 110 of the server system 100 then performs processing of storing information acquired from the terminal device 200 in the storing unit 120. For example, the storing unit 120 stores a database including body movement information and a response content as elements, and the processing at Step S107 may be update processing of the database.

In this manner, if the input operation for determining a response content has been performed (Step S102: Yes) in a first terminal device that the skilled worker uses (the terminal device 200A), the acquisition unit of the information processing apparatus (for example, the communicator 130 of the server system 100) acquires first body movement information and information with which the response content is associated. In this way, with an operation performed when the skilled worker has found an abnormality as a trigger, it is possible to accumulate the body movement information in association with the response content.

At this time, the processing unit 110 may determine a first reference point based on execution timing of the input operation in the first terminal device (for example, the terminal device 200A of the skilled care giver), and cause the storing unit 120 to store body movement information in a period to be determined by the first reference point as first body movement information. As mentioned above, the execution timing of the input operation corresponds to a situation in which the skilled worker has sensed an abnormality of a care receiver when browsing the body movement information. In other words, storing body movement information in a period corresponding to the execution timing of the input operation as first body movement information can increase the probability that the first body movement information becomes information reflected with the abnormality of the care receiver. As a result, processing accuracy improvement in the calculation or the like of the similarity, which is described later, becomes possible.

FIG. 11 is a diagram illustrating a reference point. FIG. 11 is a diagram exemplifying a time change in the respiratory rate of a given user, and is a graph similar to that of FIG. 9. Herein, a case where a skilled worker has performed an input operation of determining a response on the screens illustrated in FIGS. 9 and 10 at timing T1 indicated by an arrow in FIG. 11, is considered. As a specific situation, a case where the skilled worker recognizes, as an abnormality, an increase in the respiratory rate to the extent that can be distinguished from others in a period indicated as T2 when browsing the graph in FIG. 11, and executes the input operation at the timing T1 as a result thereof, and other cases can be considered.

In this case, the processing unit 110 sets the timing T1 as a first reference point, and stores body movement information in a period set based on the first reference point in the storing unit 120 as first body movement information. The period herein may be, for example, a period having a predetermined length using the timing T1 as an endpoint. The predetermined length may be 2 to 3 hours, may be approximately 24 hours, or may be approximately 1 to 2 weeks. For example, the server system 100 stores body movement information in a period of a certain length (for example, 2 weeks or longer) including the timing T1, and may acquire first body movement information by extracting data during a predetermined period based on the timing T1 from the body movement information. The timing T1 is not limited to the endpoint in the target period, and body movement information at timing after T1 may be included in the first body movement information. In the example of FIG. 11, T1 is set as a reference, so that it is possible to increase the probability that information during the period T2 in which an abnormality occurs in the respiratory rate is included in the first body movement information. There is also a possibility that a lag between the timing when the skilled worker browses waveforms and finds an abnormality of a user and the timing when the skilled worker inputs a response by operating the terminal device 200 occurs. For example, situations that determining a response content requires time, and an input cannot be instantly made because of another work (for example, a response and the like to an incident), and the like, are assumed. The timing T1 that is used for setting the first reference point may be correctable. The skilled worker may manually make the correction herein. Alternatively, based on the elapsed time and the like from the timing when body movement information is presented to the skilled worker to the timing T1 when the input operation is actually performed, the processing unit 110 may automatically make the correction to the timing T1.

On the other hand, if the input operation by the skilled worker has not been performed (Step S102: No), and if a response content is “NO ABNORMALITIES” (Step S103: Yes) while the input operation has been performed, the processing at Steps S104-S106 is omitted. For example, the terminal device 200 may transmit body movement information with no response content associated to the server system 100. No response content associated may indicate that a response flag is not assigned, or may indicate that all the response flags are set to the second bit (0). At Step S107, the server system 100 then performs processing of causing the storing unit 120 to store body movement information with no response content associated. Although the case where the input operation itself has not been performed and the case where “NO ABNORMALITIES” has been selected are regarded as being similar in FIG. 7, but these may be stored in distinguishable modes. For example, flag data indicating whether “NO ABNORMALITIES” has been selected may be assigned to the body movement information.

At Step S108, the processing unit 110 determines whether the measurement processing in the measuring device 400 has been ended. The determination processing at Step S108 may be executed in the measuring device 400 and the terminal device 200. If the measurement processing is not ended, the processing is returned to Step S101, and the abovementioned processing is continued. If the measurement processing has ended, the accumulation processing of data illustrated in FIG. 7 is ended.

The processing described in the foregoing executable at any timing. For example, measuring body movement information, and displaying the measurement result in the terminal device 200 may be executable all the time. The skilled care giver checks a measurement result as appropriate during the care assistance work, and executes the input operation in a case where the skilled care giver has sensed the abnormality (Step S102: Yes). In this way, every time when the skilled worker finds an abnormality and performs the input operation, body movement information and a response content are added to the database, so that it is possible to increase the amount of data that can be referred in the use processing, which is described later.

The information to be stored in the database in the embodiment may be information in which a response content is reliable to some extent. The server system 100 may store information indicating whether a plurality of care givers are respectively skilled workers. The processing unit 110 of the server system 100 may add, on the condition that data including a response content is transmitted from the terminal device 200 of the skilled worker, the data to the database. For example, data transmission to the server system 100 may be permitted only for the terminal device 200 of the skilled worker. Alternatively, while the data transmission from the terminal device 200 is permitted independent of the degree of proficiency, the server system 100 may determine whether the response content is added to the database. In this way, information indicating a response by an unskilled worker can be prevented from being used in the use processing, which is described later.

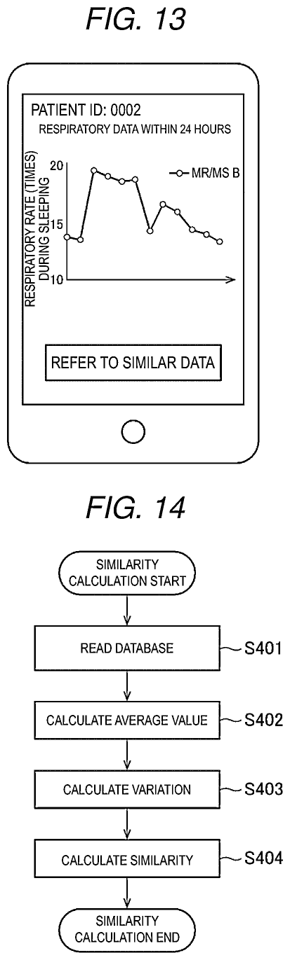

An input of whether a care giver is a skilled worker or an unskilled worker may be possible by a person in charge of the management in the care facility or the care giver himself/herself. Alternatively, the server system 100 may acquire information on a business career of each care giver, a qualification held by each care giver, and the like, and determine a degree of proficiency of the care giver based on the information. As is described later in the second embodiment, in a case where the correction of the database by a data administrator is possible, processing of correcting the degree of proficiency of each care giver in accordance with the number of times of the correction and the correction content may be performed. In a case where the correction of a database by a data administrator is possible, whether the transmitted data is added to the database may be determined based on an input operation by the data administrator. Alternatively, both of the treatment report button illustrated in FIG. 9 and a similar data reference button illustrated in FIG. 13 may be presented to a care giver who is a determination target of the degree of proficiency. The processing unit 110 then determines a degree of proficiency based on the number of times the similar data reference button is selected and/or the frequency at which the similar data reference button is selected. Selecting the similar data reference button indicates that a response content cannot be suitably determined, and thus, for example, the processing unit 110 may determine that the target care giver is an unskilled worker in a case where the number of times the similar data reference button is selected and/or the frequency at which the similar data reference button is selected are equal to or more than predetermined values. The processing unit 110 may determine a degree of proficiency based on a rate of the numbers of time each of the treatment report button and the similar data reference button are selected and/or the frequencies at which the treatment report button and the similar data reference button are selected.

### <Use Processing>

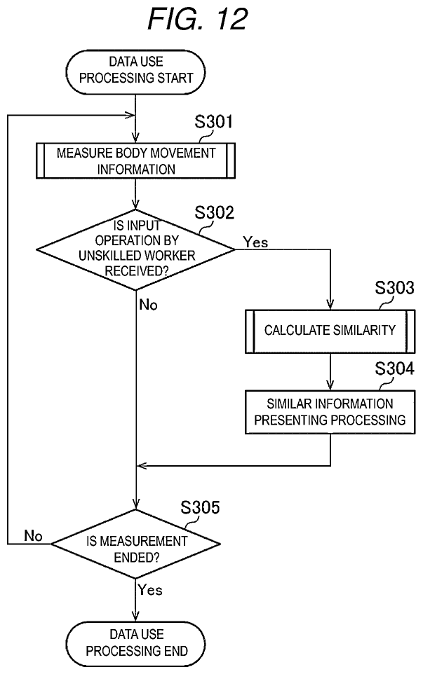

FIG. 12 is a flowchart illustrating use processing of using information accumulated in a database. Firstly, at Step S301, the detection device 430 performs a measurement of body movement information related to a target care receiver. Processing at Step S301 is similar to the processing mentioned above using FIG. 8. The detection device 430 transmits body movement information serving as a measurement result to the terminal device 200 of a care giver who is a person in charge of the care receiver.

At Step S302, the terminal device 200 executes processing of displaying body movement information on the display 240, and determination as to whether an input operation by a care giver (unskilled worker) has been received.

FIG. 13 illustrates an example of a screen to be displayed in the display 240 of the terminal device 200 at Step S302. The display 240 displays an ID for specifying a care receiver to be measured, and displays a measurement result related to the care receiver. FIG. 13 illustrates an example in which a graph indicating a change in the respiratory rate is displayed, similar to FIG. 9. The care giver who uses the terminal device 200 determines whether an abnormality is present in the care receiver by browsing the screen and the like illustrated in FIG. 13. Other body movement information such as sleep information and the heart rate may be displayed, similar to the example of FIG. 9.

An object of requesting to present body movement information and the like similar to body movement information being browsed may be displayed in the display 240 of the terminal device 200. The object herein may be, for example, the similar data reference button illustrated in FIG. 13. For example, even if a care giver with a low degree of proficiency determines that an abnormality occurs in a care receiver based on the transition of the body movement information, there is a possibility that the care giver cannot determine a specific response. In this case, the care giver may request support for the response determination of the server system 100 by performing a selection operation of the similar data reference button.

If the selection operation of the similar data reference button has been performed (Step S302: Yes), the terminal device 200 transmits a request including body movement information to the server system 100. Based on the request, at Step S303, the server system 100 performs processing of calculating a similarity.

FIG. 14 is a flowchart illustrating similarity calculation processing at Step S303. Firstly, at Step S401, the processing unit 110 performs processing of reading a database stored in the storing unit 120. The database herein is a database that is updated by the abovementioned accumulation processing, and accumulates information in which the first body movement information and the response content have been associated with each other. For example, the processing unit 110 may read all the data stored in the database at the time point of Step S401. The processing unit 110 may read part of the data in the database. For example, as is described later in the second embodiment, checking and updating the database by the data administrator may be possible, and the processing unit 110 may read only the checked data in the database.

Next, the processing unit 110 performs comparison processing between body movement information (first body movement information) that is included in the read data and body movement information (second body movement information) transmitted from the terminal device 200 of the unskilled worker, thereby calculating a similarity. For example, in a case where only N pieces (N is an integer of 2 or larger) of first body movement information have been read at Step S401, the processing unit 110 executes the following processing for each of the N pieces of the Firstly, the processing unit 110 determines a range serving as a target of the comparison processing with the first body movement information, in the body movement information transmitted from the terminal device 200 of the unskilled worker. Specifically, in a case where the processing unit 110 has acquired a request based on a second input operation in a second terminal device (the terminal device 200B in the example of FIG. 5) that the unskilled worker uses, the processing unit 110 may determine a second reference point based on execution timing of the second input operation, and determine body movement information in a period that is determined by the second reference point as second body movement information. The second input operation herein specifically indicates the selection operation of the similar data reference button in FIG. 13. For example, the processing unit 110 may set the timing when the selection operation of the similar data reference button has been performed as a second reference point, and set body movement information in a period having a predetermined length using the second reference point as an end, as second body movement information. In this way, each of the first body movement information and the second body movement information is data in which a period thereof is determined based on the input operation, so that it is possible to suitably compare the two pieces of the body movement information acquired in the different scenes with each other.

At Step S402, the processing unit 110 obtains an average value of the first body movement information and an average value of the second body movement information. For example, in a case where the body movement information is a time-series change in the respiratory rate, the average value may be an average value of the respiratory rate.

At Step S403, the processing unit 110 obtains a variation of the first body movement information and a variation of the second body movement information. For example, in a case where the body movement information is a time-series change in the respiratory rate, the variation may be a variance and a standard deviation of the respiratory rate.

At Step S404, based on the comparison between the average value of the first body movement information and the average value of the second body movement information, and the comparison between the variation of the first body movement information and the variation of the second body movement information, the processing unit 110 obtains a similarity between the first body movement information and the second body movement information. For example, the processing unit 110 may calculate a similarity based on a function with arguments of a difference between the average values and a difference between the variations. This function has a larger value as the difference between the average values is smaller, and has a larger value as the difference between the variations is smaller. The similarity may be numerical value data in a range of 0% or more and 100% or less, for example. The processing unit 110 may calculate a similarity by performing machine learning using an average value and a variation as feature data.

Steps S402-S404 indicate one example of the processing of obtaining a similarity, and the method in the embodiment is not limited thereto. For example, the processing unit 110 may calculate a similarity based on a distance between the first body movement information and the second body movement information. For example, although the example in which the difference between the average values is used for the processing has been described, instead of the average value, other values such as the maximum value, the minimum value, and the median value may be used. A changing amount of the body movement information may be used for the similarity calculation. The changing amount herein may be a difference between the maximum value and the minimum value, or may be another indicator. The changing amount may be used instead of the average value, or may be used as a simplified indicator indicating the variation.

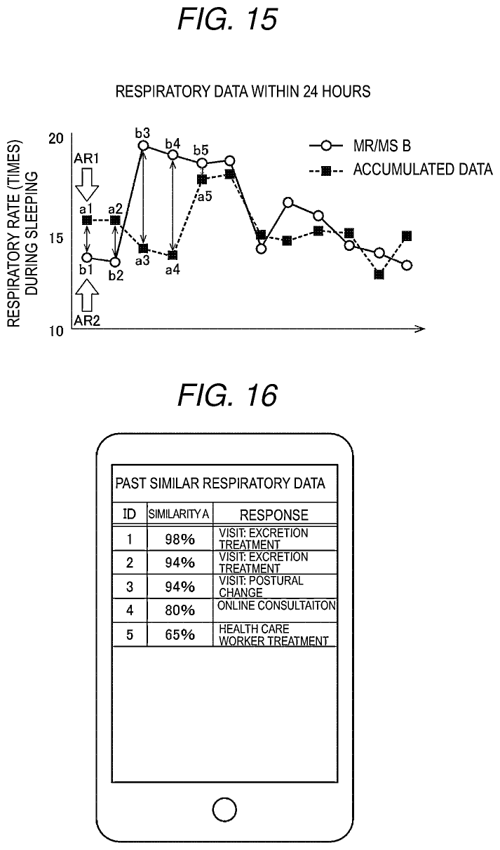

FIG. 15 is a diagram illustrating similarity calculation processing based on a distance. The measurement result (accumulated data) indicating the respiratory rate of Mr/Ms A illustrated in FIG. 9 and the measurement result indicating the respiratory rate of Mr/Ms B illustrated in FIG. 13 are collectively displayed in FIG. 15. An arrow illustrated as AR1 indicates execution timing of an input operation by a skilled care giver and indicates a first reference point. An arrow illustrated as AR2 indicates execution timing of an input operation by an unskilled care giver and indicates a second reference point. For example, the processing unit 110 may obtain, after adjusting a positional relationship between the first body movement information and the second body movement information in the horizontal axial direction so as to match the first reference point and the second reference point each other, a distance between the data at each position.

In the example of FIG. 15, the accumulated data aligns from a1 at the first reference point to a2, a3, a4, and a5 sequentially in the right direction (in the past direction in terms of time). Similarly, a measurement result of Mr/Ms B aligns from b1 at the second reference point to b2, b3, b4, and b5 sequentially in the right direction. For example, the processing unit 110 may obtain a distance d between first body movement information and second body movement information based on the following expression (1).

d={(a1−b1)²+(a2−b2)²+ . . . }^{1/2} (1)

The processing unit 110 further obtains a similarity Sr such that a value becomes larger as the distance d is smaller. For example, the similarity Sr may be obtained by the following expression (2).

Sr=1/(1+d) (2)

Various methods of obtaining a similarity between two waveforms are known, and those methods are widely applicable to the similarity calculation in the embodiment. For example, the processing unit 110 may obtain a similarity by using a cross-correlation function between the first body movement information and the second body movement information. Alternatively, the processing unit 110 may obtain a similarity by using a dynamic time warping (DTW) method.

Referring back to FIG. 12, the description is continued. With the processing at Step S303, for example, a similarity of each of the N pieces of the first body movement information read from the database with second body movement information is obtained. At Step S304, the processing unit 110 performs processing of presenting similar information including the similarity in the terminal device 200.

FIG. 16 illustrates an example of a screen to be displayed in the display 240 of the terminal device 200 by the processing at Step S304. As illustrated in FIG. 16, the processing unit 110 may perform processing of presenting a numerical value indicating the similarity and a response content associated with the first body movement information, as similar information. In FIG. 16, an ID of data with a high similarity, a numerical value indicating a similarity between the first body movement information and the second body movement information, and a response content associated with the first body movement information are displayed. As mentioned above, if the similarity between the first body movement information and the second body movement information is high, the probability that a state of a care receiver when the skilled care giver has performed the response is similar to a state of a care receiver to whom the unskilled care giver is going to perform a response is high. Accordingly, with the method in the embodiment, it is possible to present a response suitable for the state of the care receiver who is taken charge by the unskilled care giver.

In the example of FIG. 16, a response content for each data of an ID 1 and an ID 2 with a high similarity is “VISIT: EXCRETION TREATMENT”. The unskilled care giver can determine that a response of making a visit and providing excretion care assistance such as diaper changing is effective. “VISIT: POSTURAL CHANGE” is presented as a response content for an ID 3, it is possible to cause the unskilled care giver to determine that making a visit is firstly determined, and a specific response content is determined during the visit, for example.

### 1.3 Target Period of Similarity Calculation

As mentioned above, first body movement information is information that is determined based on the first reference point in a period having a given length, and second body movement information is information that is determined based on the second reference point in a period having the same length. The given length herein may be a fixed value. For example, the given length may be approximately several hours, may be 12 to 24 hours, or may be approximately 7 to 14 days. The period having a given length herein is a period when a comparison between the first body movement information and the second body movement information is made, and thus is also referred to as a comparison period in the following.

The comparison period in the embodiment is desired to be suitably set. For example, in FIG. 11, considered is a case where a comparison period is set to a period having a length including the timing T1 as the end and not including the period T2, because the comparison period is excessively short. In a case where the reason why the skilled worker has recognized an abnormality is an increase in the respiratory rate at T2, the comparison period not including T2 results in lack of information on the respiratory rate indicating the abnormality from the first body movement information. In other words, the first body movement information in this case does not include information constituting grounds for the determination of the response content by the skilled care giver. Accordingly, even if the similarity between the first body movement information and the second body movement information is high in such a case, it may not be said that the response content associated with the first body movement information is effective. In consideration of the above points, it is desired to set a comparison period to be long to such the extent that a portion (a portion where an abnormality has occurred) constituting grounds for the determination of the response content by a skilled worker is included. On the other hand, setting a comparison period to be unnecessary long may result in an increase in a processing load of the similarity calculation processing.

A preferable comparison period may change in accordance with a situation. For example, depending on a factor of the abnormality, a skilled worker can sufficiently determine a response content by browsing only several-hour measurement results in some cases, or even a skilled worker does not easily determine a response content unless he/she checks a tendency that continuously or intermittently appears in a longer period, in some cases. In other words, it can be considered that some response contents can be determined from measurement results for comparatively short time, and some response contents are not easily determined without measurement results for comparatively long time.

In consideration of the above points, the processing unit 110 may perform processing of obtaining first similarity information based on first body movement information and second body movement information in a period having a first length, and processing of obtaining second similarity information based on first body movement information and second body movement information in a period having a second length different from the first length. In this manner, the calculation accuracy improvement and the like of the similarity becomes possible by obtaining a plurality pieces of similarity information in different comparison periods.

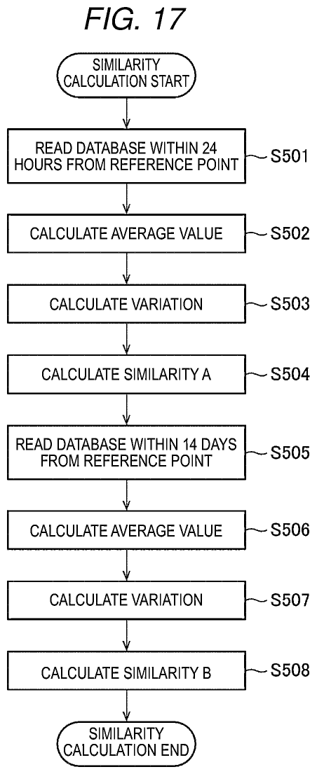

FIG. 17 is a flowchart illustrating another example of similarity calculation processing indicated at Step S303 in FIG. 12. Firstly, at Step S501, the processing unit 110 reads data including body movement information for 24 hours from the storing unit 120. For example, first body movement information to be read herein is data for 24 hours in which a period thereof is determined based on a first reference point. The processing unit 110 then calculates a similarity A by comparing the read first body movement information for 24 hours with second body movement information. The second body movement information herein is desired to be data for 24 hours in which a period thereof is determined based on a second reference point.

Processing at Steps S502-S504 is similar to the processing mentioned above using FIG. 14. For example, the processing unit 110 calculates the similarity A between the first body movement information and the second body movement information based on an average value and a variation. As mentioned above, various kinds of modifications of the processing of obtaining a similarity are executable, and various kinds of information such as the maximum value, the minimum value, a median value, and the changing amount may be used.

At Step S505, the processing unit 110 reads data including body movement information for 14 days from the storing unit 120. For example, the first body movement information to be read herein is data for 14 days in which a period thereof is determined based on a first reference point. The processing unit 110 calculates a similarity B by comparing the read first body movement information for 14 days with second body movement information. The second body movement information herein is desired to be data for 14 days in which a period thereof is determined based on a second reference point.

Processing at Steps S506-S508 is similar to the processing mentioned above using FIG. 14. For example, the processing unit 110 calculates the similarity B between the first body movement information and the second body movement information based on an average value and a variation.

For example, the processing unit 110 may present both of the similarity A and the similarity B in the terminal device 200. In this case, based on the operation input in the terminal device 200, a sort based on the similarity A, a sort based on the similarity B, and the like may be performed. In this way, a plurality of similarities from the different viewpoints are displayed to increase the information amount, so that it is possible to further support the response determination by the unskilled care giver.

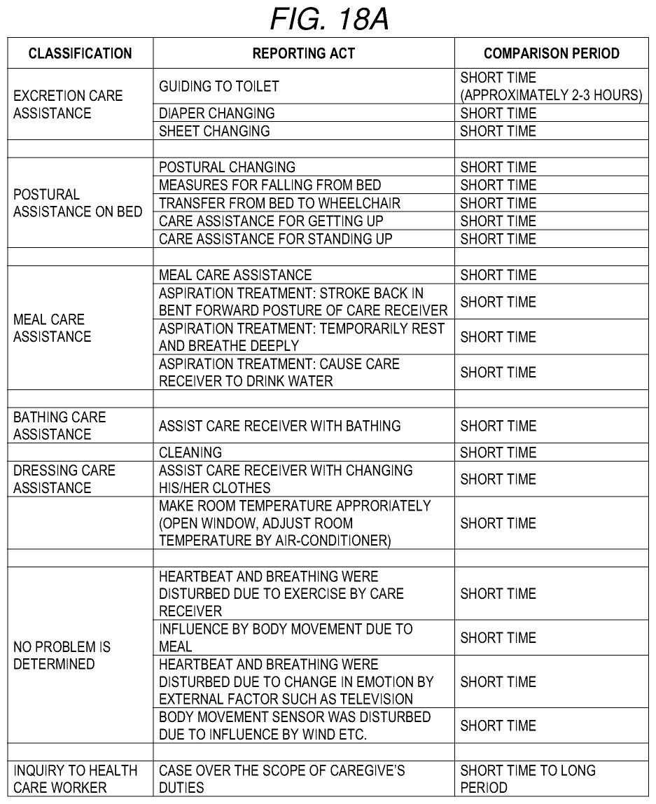

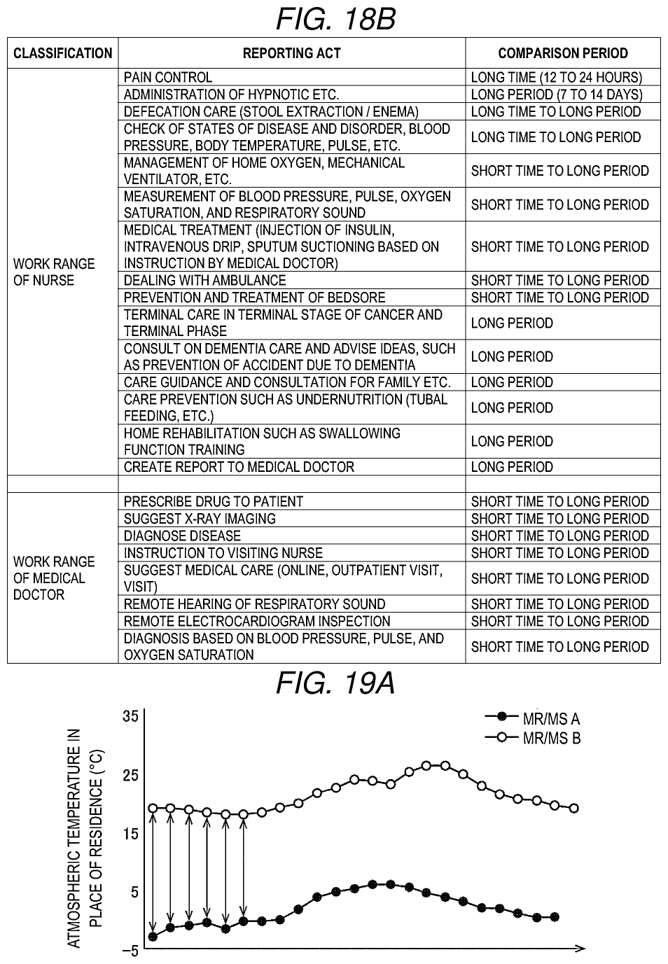

FIGS. 18A and 18B are diagrams illustrating examples of the classification of response contents, specific response contents, and desired comparison periods. FIG. 18A mainly exemplifies the response contents in a field of care assistance. For example, the excretion care assistance includes response contents such as “GUIDING TO TOILET”, “DIAPER CHANGING”, and “SHEET CHANGING”, which can be determined based on measurement results for a comparatively short period (hereinafter, expressed as for a short time) of approximately 2 to 3 hours. The same applies to postural assistance on the bed, meal care assistance, bathing care assistance, and dressing care assistance, which respectively include specific response contents, and each response content can be determined based on a measurement result for a short time. The response content of “MAKE ROOM TEMPERATURE APPRORIATELY” is not limited to that the care giver makes a visit and directly performs an air-conditioner operation and opening and closing of a window, but may include a response in which a remote operation of the air-conditioner and the like is performed using a system such as a so-called smart home. Although the adjustment of the temperature has been exemplified herein, the response content may include the adjustment of the humidity. For example, the care giver operates a humidifier and a dehumidifier directly by a visit or via a remote operation. The adjustment of the humidity may include a response in which the air-conditioner is operated in a dehumidification mode.

If no abnormalities has been determined (Step S103 in FIG. 8: Yes), there is a possibility that while a skilled worker has found an abnormality in the body movement information, the skilled worker has thought that a factor thereof can be left, or naturally resolved. For example, in a case where the reason why no abnormalities has been determined is subdivided, as illustrated in FIG. 18A, a stimulus to the care receiver due to an exercise, a meal, a television, and the like, an influence on the sensor due to wind and the like, and the like can be considered. In this case as well, the skilled worker can make a determination based on the measurement result for a short time.

In a case where it has been determined that an inquiry to a health care worker is necessary, it can be considered that the skilled care giver has determined that a response in the field of care assistance is difficult. In this case, in accordance with a response content that a health care worker actually performs based on the inquiry, sufficient characteristics is included in a measurement result for a short time in some case, and characteristics necessary for the determination of a response content is not included in some cases if a measurement result for a comparatively long time (hereinafter, expressed as a long time) of approximately 12-24 hours is not acquired. In accordance with a response by a health care worker, a longer span (hereinafter, for a long period) of approximately 7 to 14 days is necessary in some cases.

FIG. 18B is a diagram illustrating an example of response contents by health care workers and desired comparison periods. FIG. 18B separately illustrates specific examples of a work range of a nurse and a work range of a medical doctor. As a response content of a nurse, “PAIN CONTROL”, “ADMINISTRATION OF HYPNOTIC”, and the like can be considered. “MEASUREMENT OF BLOOD PRESSURE, PULSE, OXYGEN SATURATION, AND RESPIRATORY SOUND” may be performed by a visit or may be remotely performed. “DEALING WITH AMBULANCE” corresponds to a situation with high emergency, for example, atrial fibrillation and the like. “TERMINAL CARE IN TERMINAL STAGE OF CANCER AND TERMINAL PHASE” can be separated from another response, because breathing becomes gradually shallow and the respiratory rate becomes less, for example.

As a response content of a medical doctor, “PRESCRIBE DRUG TO PATIENT”, “SUGGEST X-RAY IMAGING”, and the like can be considered. The drug herein includes various kinds of drugs such as hypnotic and a sedative drug. “SUGGEST X-RAY IMAGING” may be executed in a case where there is a possibility that an influence on the body movement and the like is caused by a pain of fracture, for example. “INSTRUCTION TO VISITING NURSE” indicates that a nurse is caused to visit a patient residence, and a medical doctor remotely provides an instruction. “REMOTE HEARING OF RESPIRATORY SOUND” may be executed in a case where there is a possibility that an influence on the body movement is caused by a respiratory ailment or a swallowing trouble. Similarly, “REMOTE ELECTROCARDIOGRAM INSPECTION” may be executed in a case where there is a possibility that an influence on the body movement is caused by a cardiac disease.

For example, in a case where whether pain control, which is a response content by a nurse, is performed is determined, it is desired to acquire a measurement result for a long time. In a case where whether the administration of a hypnotic and the like is performed is determined, it is desired to acquire a measurement result for a long period. The same applies to the other response contents by a nurse, and a desired comparison period is determined for every response content. For example, in a case where dealing with an ambulance is performed, a measurement result for a short time is sufficient in some cases, and a measurement result for a long period is necessary in some cases. Two or more comparison periods of a short time, a long time, and a long period may be associated with one response content. The same applies to the work range by a medical doctor. In the first embodiment, the processing by a care giver (care person involved) is performed, so that only FIG. 18A is considered and details of FIG. 18B does not need to be considered. An example of the processing using FIG. 18B will be described later in the second embodiment.

In this manner, in a case where a response content and a desired comparison period are determined, the processing unit 110 may determine the degree of priority between first similarity information and second similarity information in accordance with a response content associated with the first body movement information.

For example, the processing unit 110 may obtain, as the similarity, a first similarity obtained by comparing body movement information for a short time, a second similarity obtained by comparing body movement information for a long time, and a third similarity obtained by comparing body movement information for a long period. The number of similarities is not limited to three, but may be two as illustrated in FIG. 17. The number of similarities may be four or more.

The processing unit 110 determines the degree of priority among the first similarity to the third similarity in accordance with a response content associated with the first body movement information. For example, in a case where the response content associated with the first body movement information is “GUIDING TO TOILET”, the comparison period for a short time is sufficient, so that the degree of priority of the first similarity is set higher than those of the second similarity and the third similarity. For example, the processing unit 110 may present the first similarity as the similarity between the first body movement information and the second body movement information. Alternatively, the processing unit 110 may perform processing of obtaining a weighted average among the first similarity to the third similarity such that the weight of the first similarity is large, and presenting the weighted average as the similarity between the first body movement information and the second body movement information.

Meanwhile, in a case where the response content associated with the first body movement information is “INQUIRY TO HEALTH CARE WORKER”, the comparison period is widely ranging from for a short time to for a long period. For example, the processing unit 110 may set the degree of priority of the third similarity higher than those of the first similarity and the second similarity, by considering that data for a long period includes data for a short time. For example, the processing unit 110 may present the third similarity as the similarity between the first body movement information and the second body movement information. Alternatively, the processing unit 110 may perform processing of obtaining a weighted average among the first similarity to the third similarity such that the weight of the third similarity is large, and presenting the weighted average as the similarity between the first body movement information and the second body movement information. Alternatively, the processing unit 110 may set the degree of priority among the first similarity to the third similarity, by considering that the comparison period is widely ranging from for a short time to for a long period.

### 1.4 Environment Information, Emotion Information

As a measurement result, body movement information based on the detection device 430 has been mainly described in the foregoing. As mentioned above using FIGS. 5, 6, and the like, the measuring device is not limited to the detection device 430, but may include the thermometer 410, the hygrometer 420, the imaging device 440, and the like. Hereinafter, the processing using these devices will be

### <Environment Information>

For example, in a case where a care receiver is an elderly person, it can be considered that the respiratory rate increases due to such factors that the care receiver has a weakened physical condition due to an abrupt change in the atmospheric temperature and suffers from a heat attack in the residence. In other words, the abnormality in the respiratory rate and the like is not limited to that generated due to some diseases, but may be caused by a surrounding environment of the care receiver. The care giver may check environment information including the temperature to be detected by the thermometer 410 and the humidity to be detected by the hygrometer 420, which are disposed in the care receiver residence, and determine a response content based on the environment information. For example, in a case where a skilled care giver has found an abnormality in the environment information, the skilled care giver may perform a response of visiting a care receiver residence, starting the air-conditioner, opening the window to provide ventilation, and the like. If the possibility that the environment information is used for the determination by the skilled worker in this manner is taken into consideration, the environment information may be used for presenting similar information to an unskilled care giver as well.

The specific processing is similar to the processing based on the body movement information. For example, the acquisition unit (for example, the communicator 130) of the information processing apparatus acquires environment information indicating a surrounding environment from an environment detection device disposed in the surrounding of a care receiver who receives at-home care. The environment detection device includes at least one of the thermometer 410 and the hygrometer 420. The environment detection device may include an illuminometer that detects the illumination, a sound volume measuring instrument that detects the loudness of sound, and the like. In other words, the environment information can include various kinds of information, such as the temperature, the humidity, the illumination, and the sound. For example, the server system 100 acquires body movement information and environment information, together with an operation result of the terminal device 200 (the terminal device 200A) by the skilled worker in the accumulation processing. In the use processing, the server system 100 acquires a request including body movement information and environment information by an operation of the terminal device 200 (the terminal device 200B) by an unskilled worker as a trigger.

The processing unit 110 may determine similar information based on the similarity between the first body movement information and the second body movement information, and the similarity between environment information associated with the first body movement information and environment information associated with the second body movement information.

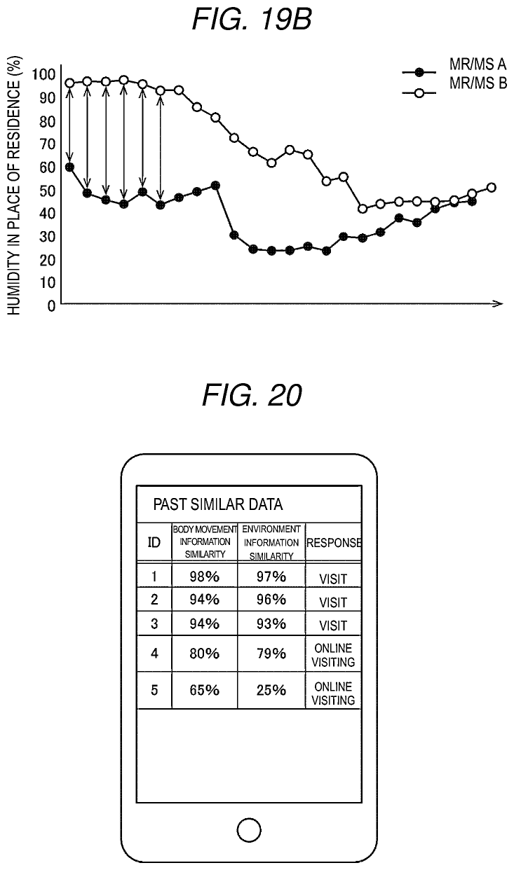

FIG. 19A is a diagram illustrating a time change in the temperature included in environment information. The horizontal axis in FIG. 19A represents time, and illustrates past data toward the right further. The longitudinal axis in FIG. 19A represents values of the atmospheric temperature. In FIG. 19A, Mr/Ms A is a care receiver who is taken charge by a skilled worker, and FIG. 19A illustrates a result in which a position of a first reference point is adjusted to the left end of the graph. Similarly, Mr/Ms B is a care receiver who is taken charge by an unskilled worker, and FIG. 19A illustrates a result in which a position of a second reference point is adjusted to the left end of the graph. For example, as illustrated in FIG. 19A, the processing unit 110 may obtain a similarity related to the temperature based on a distance between two waveforms at the corresponding positions. Various kinds of modifications can be made to the processing of obtaining a similarity, which is similar to the body movement information.

FIG. 19B is a diagram illustrating a time change in the humidity included in environment information. The horizontal axis in FIG. 19B represents time, and illustrates past data toward the right further. The longitudinal axis in FIG. 19B represents values of the humidity. Similarly in the example of the humidity, the processing unit 110 may obtain a similarity related to the humidity based on a distance between two waveforms.

In this way, the processing unit 110 can respectively obtain a similarity related to body movement information and a similarity related to environment information. For example, the processing unit 110 may perform processing of displaying the two similarities in association with the response content in the terminal device 200.

FIG. 20 illustrates an example of a screen to be displayed in the display 240 of the terminal device 200. As illustrated in FIG. 20, the processing unit 110 may perform processing of presenting, as similar information, an ID of data with high similarities, a numerical value indicating the similarity of the body movement information, a numerical value indicating the similarity of the environment information, and a response content associated with the first body movement information. In this way, the degree of similarity of the environment information is presented as well, so that it is possible to more suitably support the response determination by the unskilled care giver.

As mentioned above, in a case where the similarity of the temperature and the similarity of the humidity are obtained, these both similarities may be presented, or one similarity related to the environment information may be obtained based on these two similarities. The same applies to the body movement information, for example, in a case where the similarity of the respiratory rate and the similarity of the heart rate have been respectively obtained, these both similarities may be presented, or one similarity related to the body movement information may be obtained based on these two similarities.

One similarity may be obtained based on the similarity of the body movement information and the similarity of the environment information. For example, the processing unit 110 may respectively obtain the similarity of the respiratory rate, the similarity of the heart rate, the similarity of the temperature, and the similarity of the humidity, and obtain one similarity based on these similarities. The similarity herein may be a result by multiplying the four similarities, or may be a result of obtaining a weighted average of the four similarities. The same applies to a case where body movement information other than the respiratory rate and the heart rate is used, and a case where environment information other than the temperature and the humidity is used.

### <Emotion Information>

The acquisition unit of the information processing apparatus may acquire, based on image information in which a user who receives at-home care (care receiver) has been imaged, emotion information indicating an emotion of the user. For example, as a method of estimating an emotion of a person based on an image, machine learning based on training data in which relative to input data that is an image obtained by imaging a face of a person, information specifying an emotion of the person is assigned as ground truth data, may be used. For example, the processing unit 110 performs an emotion analysis by inputting an imaged image acquired from the imaging device 440 into a learned model acquired by the machine learning. The model herein is, for example, CNN (Convolutional Neural Network). Various kinds of methods of analyzing an emotion based on image processing, such as Face API disclosed in “https://azure.Microsoft.com/ja-jp/services/cognitive-services/face/” are known, and those methods are widely applicable in the embodiment. The emotion information herein is information specifying which one is an emotion of a care receiver among a plurality of emotions of happiness, sadness, anger, fear, surprise, antipathy, neutrality, and the like. Hereinafter, an example in which the processing unit 110 of the server system 100 performs an emotion analysis will be described, but another device such as the terminal device 200 may execute the emotion analysis.

For example, the processing unit 110 estimates an emotion change of a care receiver because a response indicated by a response content has been performed based on emotion information associated with first body movement information, and labels the first body movement information based on the estimation result.

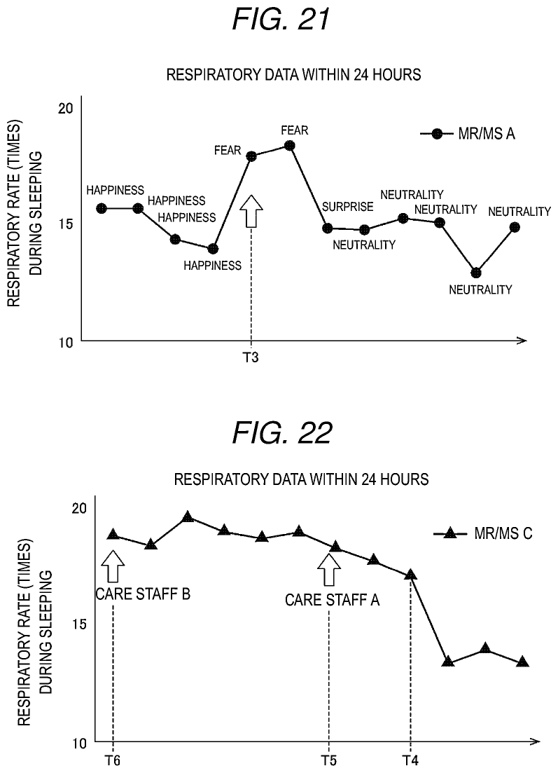

FIG. 21 is a graph in which a result of an emotion analysis at each timing is associated with a time change in body movement information. For example, the longitudinal axis represents the respiratory rate, and the horizontal axis represents time. Similar to the abovementioned example, the right side on the horizontal axis represents past. In the example of FIG. 21, at timing T3 indicated by an arrow, an input operation by a care giver was performed, and a visit to a care receiver residence as a specific response was executed. As illustrated in FIG. 21, while the emotion of the care receiver was neutral in a case where the respiratory rate was a usual value (approximately 15 times/minute) at the graph right side, the emotion of the care receiver changes to fear when an abnormality occurs and the respiratory rate increases. In other words, the neutral emotion changes to the negative emotion due to the abnormality occurrence. Thereafter, the care giver visits the care receiver residence and performs a response, so that the respiratory rate becomes stable and the emotion of the care receiver has changed to a positive emotion of happiness.

In this manner, in a case where the emotion of the care receiver has changed from the negative to the positive direction, it is determined that the response by the care giver has been effective. In this case, the processing unit 110 may assign a label indicating success data to the target data. The positive emotion indicates happiness, and the negative emotion indicates sadness, anger, fear, surprise, antipathy, and the like. Neutrality indicates an intermediate emotion between positive and negative emotions.

On the other hand, in a case where the emotion of the care receiver has changed from the positive to the negative, it is determined that the response by the care giver has not been effective. In this case, the processing unit 110 may assign a label indicating failure data to the target data. The processing unit 110 may assign a label indicating neutral data to the target data in a case where the emotion of the care receiver has not been changed.

For example, data labeled with success data indicates that the response content by the care giver is preferable, and thus is desired to be a target to the presenting processing to an unskilled worker. On the other hand, data labeled with failure data indicates that the response content by the care giver has been not preferable, thus, there is a possibility that in a case where the response content included in the failure data is presented, an unskilled worker is prompted to execute the unpreferable response.

When the database is read (Step S401 in FIG. 14 and the like) in the similarity calculation processing, the processing unit 110 may perform processing of reading only data labeled with success data in the database. Alternatively, the processing unit 110 may perform processing of reading the data excluding failure data (data labeled with success data and neutral data) in the database.

Although the processing using an emotion change caused by the response execution has been described in the foregoing, the use of the emotion information is not limited thereto. For example, in a case where an abnormality has occurred in a body movement of a care receiver, as one of factors thereof, an emotion change of the care receiver can be considered. For example, there is a possibility that the respiratory rate and the heart rate increase by such a factor that the care receiver experiences a negative emotion such as anger and sadness. For example, in the terminal device 200 of a care giver who is a skilled worker or an unskilled worker, emotion information is presented together with body movement information, so that it is possible to support the determination of a response content by the care giver. For example, in the screens of FIGS. 9 and 13, emotion information may be displayed. When similar information is determined, comparison processing between emotion information associated with the first body movement information and emotion information associated with the second body movement information may be performed. For example, the processing unit 110 may perform processing of labeling emotion before and after an abrupt change of the first body movement information with the first body movement information and processing of labeling emotion before and after an abrupt change of the second body movement information with the second body movement information, and perform comparison processing of the two labels. In this way, it is possible to cause the care giver to suitably perform a response to the abnormality caused by the emotion.

Labeling an emotion as mentioned above is especially preferable when a response of calming the emotion is executed. For example, among the response contents illustrated in FIG. 18A, in “ASPIRATION TREATMENT: STROKE BACK IN BENT FORWARD POSTURE OF CARE RECEIVER”, “ASPIRATION TREATMENT: TEMPORARILY REST AND BREATHE DEEPLY”, “ASPIRATION TREATMENT: CAUSE CARE RECEIVER TO DRINK WATER”, and the like, it is important to cause the care receiver to calm down and to calmly perform swallowing, so that there is a possibility that care assistance can be effectively executed by considering the emotion when the aspiration has occurred. “ASSIST CARE RECEIVER WITH BATHING”, “CLEANING”, and the like allow the care receiver to change his/her mood, and are thus effective responses to an abnormality caused by the emotion. In a case where there is a possibility that these responses are presented, for example, in a case where these response contents are associated with first body movement information serving as a comparison target with second body movement information, the processing unit 110 may perform the similarity calculation processing by positively using a labeling result of the emotion. For example, the processing unit 110 may use the emotion labeling result in a case where these response contents are associated, and does not need to use the emotion labeling result in another response content. Alternatively, the processing unit 110 may set the degree of priority of the emotion labeling result higher in a case where these response contents are associated than in a case where another response content is associated.

Among “NO ABNORMALITIES IS DETERMINED”, as for “HEART RATE AND BREATHING WERE DISTURBED DUE TO EXERCISE BY CARE RECEIVER”, “INFLUENCE BY BODY MOVEMENT DUE TO MEAL”, “HEART RATE AND BREATHING WERE DISTURBED DUE TO CHANGE IN EMOTION BY EXTERNAL FACTOR SUCH AS TELEVISION”, and the like, since an abnormality in the body movement information is not caused by a disease and the like, the degree of influence by the emotion is large. When the processing related to these response contents is performed as well, a labeling result of the emotion may be used.

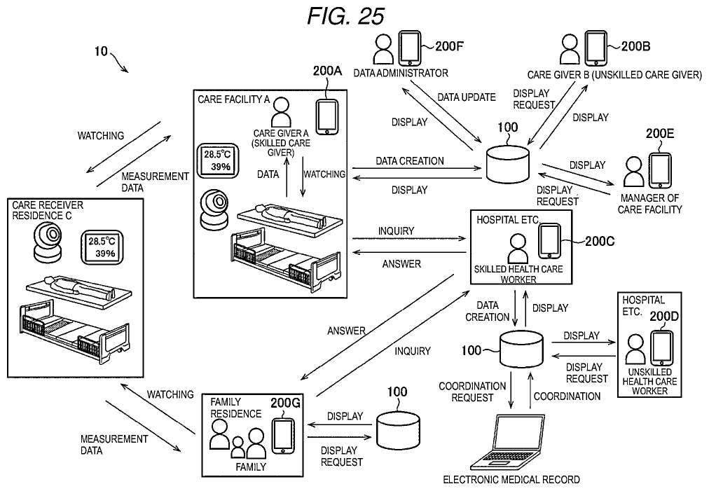

In addition, in a case where a response content by a health care worker is considered, a labeling result of the emotion may be used in the separation of an abnormality factor (determination of a response content). For example, there is a possibility that a nurse may use an emotion of a patient, as a determination reference for selecting any of “PAIN CONTROL”, “DEFECATION CARE”, “CHECK OF STATES OF DISEASE AND DISORDER, BLOOD PRESSURE, BODY TEMPERATURE, PULSE, ETC.”, and “MANAGEMENT OF HOME OXYGEN, MECHANICAL VENTILATOR, ETC.”, among the response contents illustrated in FIG. 18B. In a case where the processing unit 110 displays body movement information in the terminal device 200 (the terminal device 200C or 200D, which is described later in FIG. 25 in second embodiment) of a health care worker, the processing unit 110 may display emotion information together. As is described later, similar information may be presented to an unskilled health care worker, and emotion information may be used in similarity calculation processing therefor.

### 1.5 Correction of Reference Point

The example in which the execution timing of the input operation by the skilled care giver is set as the first reference point, and the execution timing of the input operation by the unskilled care giver is set as the second reference point has been described in the foregoing. For example, as mentioned above using FIG. 15 and the like, after the positions are adjusted so as to match the first reference point and the second reference point each other, the comparison processing between the first body movement information and the second body movement information is executed.

If care givers browse the same measurement result, each of the care givers finds an abnormality from the measurement result at different timing. For example, the care giver with a high degree of proficiency can find an abnormality at early timing, but the care giver with a low degree of proficiency takes time before the care giver notices the abnormality in some cases.

FIG. 22 is a diagram illustrating an example of a difference in execution timing of the input operations by care givers. In FIG. 22, the longitudinal axis represents the respiratory rate, and the horizontal axis represents time (past is in the right direction). For example, after the respiratory rate has increased at timing T4, a skilled worker finds an abnormality and executes the input operation at timing T5 with a comparatively short elapsed time. On the other hand, an unskilled worker cannot easily notice an abnormality after the respiratory rate has increased, and finds the abnormality and executes the input operation at timing T6 after a comparatively long time has elapsed.

In a case where the timing of the input operation differs between the skilled worker and unskilled worker in this way, the time from the abnormality occurrence to the reference point is different, so that even if the measurement results are compared with each after the first reference point and the second reference point are corrected so as to match each other, there is a possibility that the calculation accuracy of the similarity is lowered.

In the embodiment, correction processing of a reference point may be performed. For example, the processing unit 110 may perform processing of correcting the first reference point based on the changing amount of the first body movement information per unit time. Similarly, the processing unit 110 may perform processing of correcting the second reference point based on the changing amount of the second body movement information per unit time.

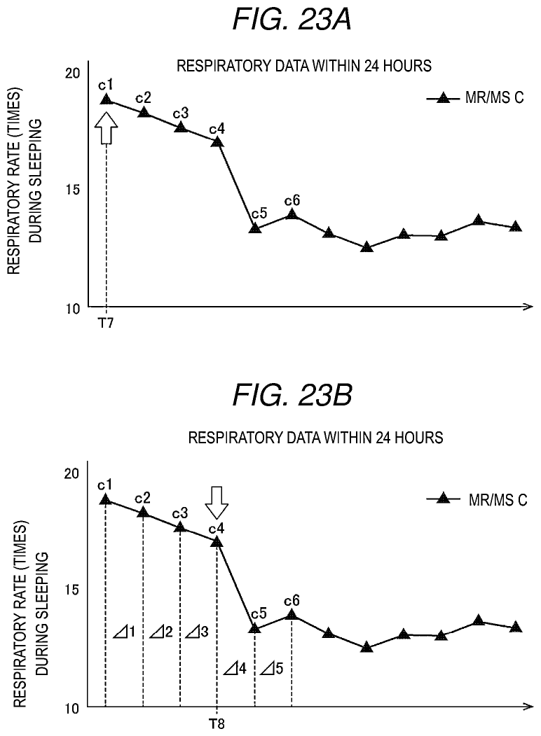

FIG. 23A is a diagram exemplifying a reference point before the correction processing. In FIG. 23A, the longitudinal axis represents the respiratory rate, and the horizontal axis represents time (past is in the right direction). The content of the correction processing of a reference point is common between the first reference point and the second reference point, and the description will be made hereinafter simply using a reference point. For example, the respiratory rate is stable at a low value until timing c5, but increases to the extent that can be identified at and after c4. A care giver has found an abnormality at timing T7 corresponding to c1, and has performed an input operation in the terminal device 200. In this case, the timing T7 is set as a reference point before the correction.

FIG. 23B is a diagram illustrating a content of correction processing and a reference point after the correction processing. For example, the processing unit 110 obtains a difference (differentiation) in the respiratory rate at two adjacent timings, and performs comparison processing between the difference and a given threshold. In FIG. 23B, A1 to 45 each indicates a difference, and Δ1=|c1−c2| is obtained. The same applies to Δ2 to Δ5. Instead of the difference, the rate between two adjacent numerical values may be used.

In the example of FIG. 23B, a value of Δ4 is larger than the threshold, and values of Δ1 to Δ3 and Δ5 are each smaller than the threshold. In this case, the processing unit 110 performs processing of correcting the reference point at timing of c4 (timing T8) when a change equal to or more than the threshold has occurred. In this way, the reference point can be corrected in accordance with the specific change in the body movement information, so that the difference between the first reference point and the second reference point caused by the timing of the input operations illustrated in FIG. 22 is reduced. As a result, the accuracy improvement in the similarity calculation processing between the first body movement information and the second body movement information becomes possible.

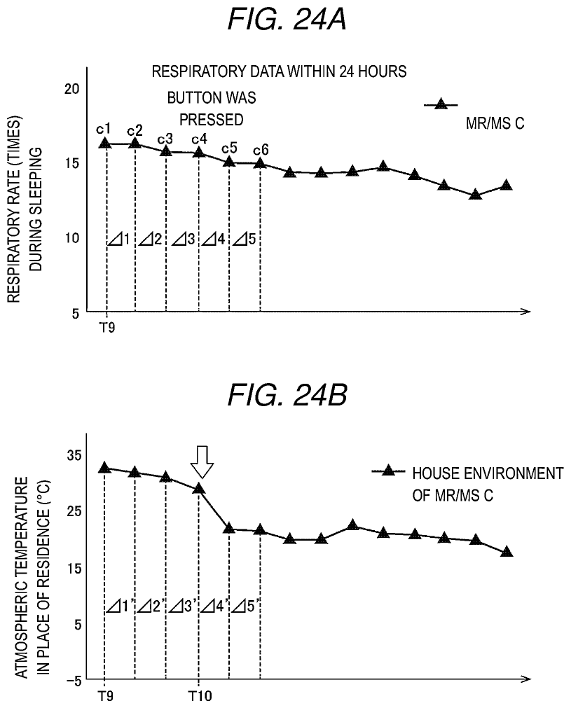

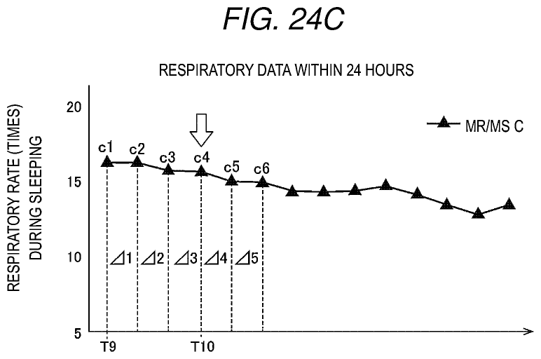

In some measurement results, a point where a difference becomes equal to or more than the threshold cannot be found in some cases. FIGS. 24A to 24C are other diagrams illustrating correction processing of a reference point. FIG. 24A is a diagram illustrating a time change in the respiratory rate. In this case, the respiratory rate gently increases. Accordingly, even if 41 to 45 are obtained similar to FIG. 23B, no point exceeding the threshold is present, so that the reference point cannot be corrected from timing T9 that is execution timing of the input operation.

In such a case, the processing unit 110 may perform processing of correcting the first reference point and the second reference point based on the changing amount in the environment information per unit time. FIG. 24B is a diagram illustrating environment information of the same target care receiver as the body movement information in FIG. 24A acquired in the same period. FIG. 24B exemplifies a time change in the temperature in the environment information. In this case as well, similarly, the processing unit 110 obtains a difference (differentiation) or a rate of temperatures at two adjacent points, and searches a point at which the difference or the rate becomes equal to or more than the threshold. In the example of FIG. 24B, it is determined that Δ4′ is equal to or more than the threshold, among Δ1′ to Δ5′ that are differences. The processing unit 110 performs processing of correcting the reference point to timing T10 at which the temperature change equal to or more than the threshold has occurred.

The processing unit 110 performs processing that uses the reference point corrected by using the environment information also when performing the comparison processing between the first body movement information and the second body movement information. FIG. 24C is a diagram illustrating a time change in the respiratory rate, similar to FIG. 24A. In this case, the processing unit 110 sets timing T10 illustrated in FIG. 24B as a reference point. In this way, even in a case where the time change in the body movement information is small, the reference point can be suitably corrected, so that the accuracy improvement in the similarity calculation processing becomes possible.

### 1.6 Modification Example of Similarity Calculation Processing

Hereinafter, a further modification example of the processing of calculating a similarity will be described. For example, a skilled worker determines an abnormality using a standard range and a sudden change of body movement information as a reference. By calculating a similarity based on the standard range or the sudden change of the body movement information, suitable processing reflected with tacit knowledge of the skilled worker can be executed.

### <Standard Range>

For example, the processing unit 110 may extract part or all of first body movement information and second body movement information based on the standard range of the body movement information, and calculate a similarity by the comparison processing of extraction results. The standard range is information indicating a numerical value range of body movement information for an average care receiver. For example, the body movement information is sleep information, e specifically may be the respiratory rate. The standard range of the respiratory rate is, for example, approximately 12-24 times per one minute. It is needless to say that the body movement information is not limited to the respiratory rate, but can be replaced with another information such as the heart rate. Hereinafter, for simple description, processing in which an upper limit (24 times) of the standard range is used will be described, but similar processing may be performed for the lower limit.

The processing unit 110 extracts data out of the abovementioned standard range, in the body movement information. For example, the processing unit 110 extracts, in the first body movement information illustrated in FIG. 15, a portion in which the respiratory rate is more than 24 times as first comparison information. Similarly, the processing unit 110 extracts, in the second body movement information, a portion in which the respiratory rate is more than 24 times as second comparison information.

The processing unit 110 then calculates a similarity based on the comparison between the number of points included in the first comparison information and the number of points included in the second comparison information, in the graph such as FIG. 15. As the numbers of points are closer to each other, the similarity is calculated higher. In this way, it is possible to calculate a similarity based on the degree of deviation from the standard range. In other words, it is possible to reflect the determination reference by the skilled worker to the calculation processing of the similarity.

The processing unit 110 may calculate a similarity based on a distance between the first comparison information and the second comparison information. The similarity calculation processing based on the distance is similar to the abovementioned example using FIG. 15. At a given position on the horizontal axis, in a case where the first body movement information is out of the standard range but the second body movement information is within the standard range, second comparison information is not present at the position. In this case, the processing unit 110 may use, instead of a distance between first comparison information and second comparison information, a distance between the first comparison information and the upper limit value (24 times) of the standard range. The same applies to a case where second comparison information is present, and first comparison information is not present.

The processing unit 110 may obtain an area of a range surrounded by a polygonal line (graph) indicating first comparison information and a straight line in transverse direction indicating an upper limit value (24 times) of the standard range, as an indicator value for comparison. Similarly, the processing unit 110 obtains an area of a range surrounded by a polygonal line indicating second comparison information and a straight line in transverse direction indicating an upper limit value of the standard range, as an indicator value for comparison. The processing unit 110 may then determine that as the two indicator values are closer to each other, the similarity is higher.

The processing unit 110 may calculate indicator values such as an average value, a maximum value, a minimum value, and a variation, relative to the first comparison information. Similarly, the processing unit 110 calculates indicator values such as an average value, a maximum value, a minimum value, and a variation, relative to the second comparison information. The processing unit 110 may then determine that as a difference between the indicator value calculated from the first comparison information and the indicator value calculated from second comparison information is closer, the similarity is higher.

### <Sudden Change in Value>

The processing unit 110 may extract part or all of first body movement information and second body movement information based on a point at which a value of the body movement information changes by equal to or more than a predetermined value, and calculate a similarity by comparison processing of extraction results. For example, the processing unit 110 obtains a difference (differentiation) between two adjacent points in the first body movement information, and determines a point at which the value of the difference becomes equal to or more than a predetermined threshold as a point at which the first body movement information suddenly changes. The processing of detecting a point at which the value suddenly changes may be the same as the processing mentioned above using FIG. 23B. The processing unit 110 then extracts first body movement information in a range from a first reference point as one end to a point the closest to the first reference point at which a value suddenly changes as the other end, as first comparison information. The same applies to second body movement information, and the processing unit 110 extracts second body movement information in a range from a second reference point as one end to a point the closest to the second reference point at which a value suddenly changes as the other end, as second comparison information. In this way, based on the point at which the value serving as a determination reference by the skilled worker suddenly changes, it is possible to set a target range for similarity calculation.

The processing after the first comparison information and the second comparison information have been extracted is similar to the abovementioned example of the standard range. In other words, a similarity may be calculated based on the number of points included in the first comparison information and the number of points included in the second comparison information. Alternatively, a similarity may be calculated based on a distance between the first comparison information and the second comparison information. Alternatively, a similarity may be calculated based on an area between first comparison information and a given straight line (may be a straight line indicating an upper limit of the standard range or may be a straight line corresponding to another value), and an area between second comparison information and the given straight line. Alternatively, an indicator value such as an average value, a maximum value, a minimum value, or a variation is obtained from first comparison information and second comparison information, and a similarity may be calculated based on the indicator value.

### 2. Second Embodiment

FIG. 25 illustrates another example of the information processing system 10, and is a diagram illustrating a specific use case. The care facility A, the care receiver residence C, and the care giver A with a high degree of proficiency are similar to those in FIG. 5, and detailed descriptions are thus omitted. Although the illustration of the care giver B with a low degree of proficiency is omitted, for example, similar to FIG. 5, the care giver B may work for the care facility B, and perform watching of the care receiver B who occupies the care facility B and the care receiver D in the care receiver residence D.

As illustrated in FIG. 25, in addition to the configuration illustrated in FIG. 5, configurations related to a health care worker, a manager of a care facility, a data administrator, and a family of a care receiver may be added in the embodiment. The health care worker answers an inquiry from a care giver by using a terminal device 200C. The terminal device 200C may perform data transmission to the server system 100. The health care worker may include a skilled health care worker with a high degree of proficiency and an unskilled health care worker with a low degree of proficiency. The unskilled health care worker refers to data accumulated in the server system 100 by using a terminal device 200D. The server system 100 may acquire information related to an electronic medical record of a care receiver. As is understood from the description in the foregoing, a care receiver in the embodiment may be a patient who receives care (clinical examination, inspection, and the like) by a health care worker, and thus a care receiver is also referred to as a patient in the following description.

A manager of a care facility refers to data accumulated in the server system 100 resulting from the care assistance in the care facility, by using a terminal device 200E. A data administrator performs management of the data accumulated in the server system 100 by using a terminal device 200F. The data administrator is a staff of a care facility, for example, and may perform management of the data accumulated in the server system 100 resulting from care assistance in the care facility. The data administrator is not limited to a staff in the care facility, and may be a provider (a business operator who performs construction, management, and the like of a system including the server system 100) who provides service according to the embodiment.

As a family of a care receiver, a family who lives separately from the care receiver is assumed herein. For example, a family of the care receiver C lives in a family residence different from the care receiver residence C. The family acquires a measurement result related to the care receiver C and makes an inquiry to a medical doctor by using a terminal device 200G. The terminal device 200G may acquire log data related to care assistance and medical care from the server system 100.

Hereinafter, processing related to each of the health care worker, the manager of the care facility, the data administrator, and the family of a care receiver will be described.

### 2.1 Health Care Worker

The health care worker includes various kinds of experts, such as medical doctors, nurses, and pharmacists, who engage in medical care. For example, as illustrated in FIG. 25, the health care worker receives an inquiry from a care giver, and answers the inquiry. For example, in a case where the care giver has selected “INQUIRY TO HEALTH CARE WORKER” as a response, the terminal device 200C that a medical doctor uses is notified of an inquiry. The notification herein may be directly transmitted from the terminal device 200A to the terminal device 200C or may be transmitted via the server system 100.

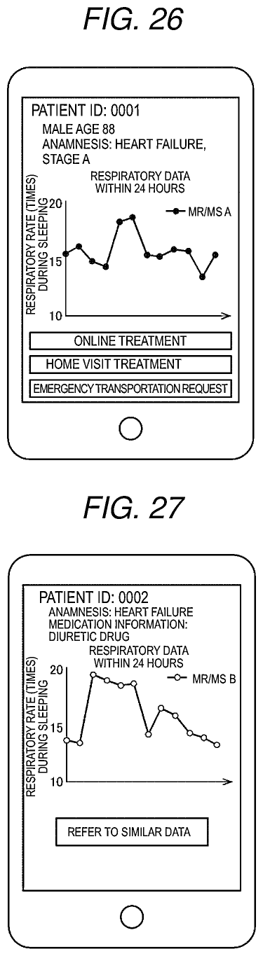

FIG. 26 illustrates an example of a screen to be displayed on the terminal device 200C of the health care worker in a case where the terminal device 200C has received the inquiry. On the screen illustrated in FIG. 26, an ID for specifying a care receiver, an attribute (gender, age, anamnesis), a measurement result, and objects for inputting a response are displayed. In the example of FIG. 26, such facts that an ID of a care receiver is 0001, and the care receiver is male at the age of 88 and has an anamnesis of heart failure are displayed. The health care worker determines a response based on the information. The information to be displayed on the screen illustrated in FIG. 26 may be transmitted from the terminal device 200 (the terminal device 200A in the example of FIG. 25) serving as an inquiry source. Alternatively, the information is accumulated in the storing unit 120 of the server system 100, and the terminal device 200C of the health care worker may acquire the information from the server system 100 in a case where the terminal device 200C has received an inquiry.

Although the three server systems 100 are illustrated in FIG. 25, these may be implemented by one server system 100, or may be distributed servers that can share data. In other words, a health care worker may be able to refer to information registered by a person involved in care assistance to the server system 100, and a care giver may be able to refer to information registered by the health care worker to the server system 100. The same applies to the examples of the manager, the data administrator, and the family of the care receiver.

FIG. 26 illustrates an example in which as objects for inputting a response, an online treatment button, a home visit response button, an emergency transportation request button are displayed. The online treatment indicates a response in which the health care worker remotely makes a diagnosis and the like. For example, similar to the example of the remote care assistance, an online treatment may be performed by using a system such as a video conference. The home visit response indicates a response in which the health care worker visits the care receiver residence (patient residence), and performs a direct response. The emergency transportation request indicates a response in which the emergency transportation of a care receiver to a hospital is requested, and is selected in a case where the state of the care receiver is bad and the urgency is high. The responses by the health care worker are not limited thereto, and an input of a more detailed response may be possible on the screen illustrated in FIG. 26. The examples of the specific response contents are as illustrated in FIG. 18B.

In a case where a health care worker has performed an input operation of selecting any of the responses, the health care worker starts the input specific response. The terminal device 200C of the health care worker transmits a response content serving as an input result to the terminal device 200A serving as an inquiry source. In this way, it is possible to present a response content that the health care worker executes to a care giver who has made the inquiry. The care giver may execute a specific action to a part of the response content based on an instruction by the health care worker.

As illustrated in FIG. 25, in a case where a health care worker who has received an inquiry is a skilled worker, the probability that the health care worker can suitably determine a response based on the information to be displayed in FIG. 26 is high. However, an unskilled worker is included in the health care workers, so that the health care worker cannot determine a specific response even if browsing the information displayed in FIG. 26 in some cases.

In the embodiment, similar to the example of the care giver in the first embodiment, the server system 100 may perform processing of accumulating data by a skilled health care worker in the storing unit 120 as accumulated data, and presenting a reference result of the accumulated data to an unskilled health care worker. In this way, even in a case where an unskilled health care worker has received an inquiry, it is possible to determine a suitable response in accordance with a state of the care receiver.

For example, in a case where a skilled health care worker has performed an input operation of selecting some response in the screen of FIG. 26, the terminal device 200C transmits the selected response content to the server system 100. The server system 100 stores the measurement result related to the care receiver in association with the response content by the skilled health care worker, in the storing unit 120.

FIG. 27 illustrates an example of a screen to be displayed, in a case where an unskilled health care worker has received an inquiry from a care giver, in the display 240 of the terminal device 200D that the unskilled health care worker uses. An ID for specifying a care receiver, an attribute, and a measurement result in FIG. 27 are similar to those in FIG. 26. In the example of FIG. 27, such facts that an ID of a care receiver is 0002, the care receiver has an anamnesis of heart failure, and a diuretic drug is prescribed are displayed. The screen of FIG. 27 may include, for example, similar to FIG. 13, a similar data reference button. In a case where a selection operation of the similar data reference button in FIG. 27 has been performed, the terminal device 200D requests the server system 100 to present similar information.

The processing unit 110 of the server system 100 reads first body movement information from the database, and obtains a similarity by comparing the first body movement information with body movement information (second body movement information) illustrated in FIG. 27. The similarity calculation processing is similar to that in the first embodiment, and the processing illustrated in FIG. 14 may be performed or the processing illustrated in FIG. 17 may be performed. Candidates for the response are presented to the health care worker herein, and thus there is a possibility that presenting a response by the skilled care giver is not useful. The data to be read at Step S401 and the like in FIG. 14 may be limited to the data associated with the response content by the skilled health care worker. In a case where the processing illustrated in FIG. 17 is performed, the degree of priority among a plurality of similarities may be determined based on “COMPARISON PERIOD” illustrated in FIG. 18B. The server system 100 transmits similar information including the similarity to the terminal device 200D.

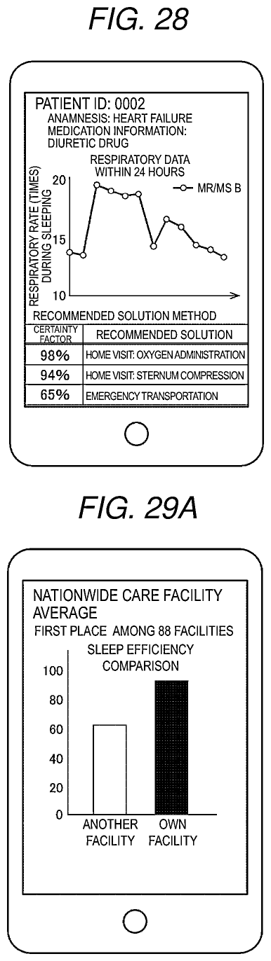

FIG. 28 illustrates an example of a display screen of similar information to be displayed in the display 240 of the terminal device 200D. An ID for specifying a care receiver, an attribute, and a measurement result in FIG. 28 are similar to those in FIG. 27. On the screen illustrated in FIG. 28, the numerical value of the similarity between the first body movement information and the second body movement information, and the response content associated with the first body movement information are displayed. In FIG. 28, an example in which machine learning is used is assumed, and the value of the certainty factor is displayed as the similarity. The example of machine learning is described later together with a description related to the data administrator. In the example of FIG. 28, response contents of “HOME VISIT: OXYGEN ADMINISTRATION”, “HOME VISIT: STERNUM COMPRESSION”, and “EMERGENCY TRANSPORTATION” are presented in descending order of the similarity. In this manner, presenting the response content together with the similarity enables the health care worker with a low degree of proficiency to perform a suitable response as well.

In the forgoing, the example in which a health care worker determines a response content using an inquiry from a care giver as a trigger has been described, but the embodiment is not limited thereto. For example, a measuring device 400 may be disposed in a sickroom of an inpatient or a home of an outpatient, and a measurement result of the measuring device 400 may be transmitted to the terminal device 200 (for example, the terminal device 200C or 200D in FIG. 25) of the health care worker. The health care worker may periodically check the measurement result, and input a response content into the terminal device 200 if determining the necessity, and execute a specific response. For example, the processing similar to that in FIG. 7 is executed in a case where a skilled health care worker input a response content, and the processing similar to that in FIG. 12 is executed in a case where an unskilled health care worker was unable to decide a response. In this way, even for a patient whose care is taken by a health care worker without a care giver, it is possible to cause the health care worker to perform a suitable response independent of the degree of proficiency.

As mentioned above, the measuring device 400 in the embodiment may include a clinical thermometer, an electrocardiogram meter, a pulse meter, a sphygmomanometer, a pulse oximeter, a body composition meter, a stethoscope, and the like. The care giver may use these devices. The health care worker may perform an action that may serve as a medical practice, such as a check of breathing sound by the stethoscope. For example, a nurse or the like may use the abovementioned measuring device 400, and a measurement result in association with first body movement information and a response content may be stored in the server system 100. In the use processing, similarly, a measurement result measured by a nurse or the like who uses the abovementioned measuring device 400 is transmitted to the server system 100, and the processing unit 110 uses the measurement result to the similarity calculation processing.

### 2.2 Manager

As illustrated in FIG. 25, the terminal device 200E that is used by a manager of a care facility may perform communication with the server system 100. For example, the terminal device 200E acquires various kinds of information including an operation history and the like of the care giver from the server system 100.

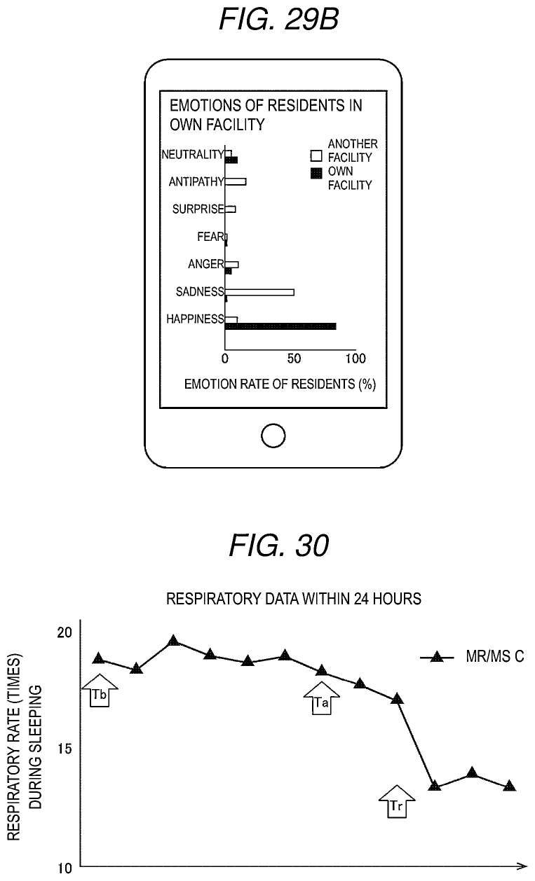

FIGS. 29A and 29B illustrate examples of screens to be displayed in the terminal device 200E. FIG. 29A is a diagram in which the sleep efficiency of care receivers who occupy the care facility is displayed. For example, the processing unit 110 of the server system 100 may obtain the sleep efficiency of each of a plurality of care receivers based on sleep information included in the body movement information. The sleep efficiency indicates a rate of the actual sleep time relative to the bed-presence time. The processing unit 110 may obtain the sleep efficiency based on a determination result of sleep or awake and a determination result of bed-presence or bed leaving. For example, the sleep efficiency is lowered if the time during when the care receiver is in bed but cannot sleep or the time during when the care receiver is awake but cannot get out from the bed, is present. The sleep efficiency is desired to be a high value to some extent (for example, 85% or more).

For example, the server system 100 may obtain a value of the sleep efficiency for every care facility. The sleep efficiency in a care facility is, for example, an average value of the sleep efficiency of all the residents who occupy the care facility. Various kinds of modifications of a method of obtaining the sleep efficiency in each care facility can be made, such as the high rate of the sleep efficiency of residents having a specific attribute. The server system 100 presents information comparing the sleep efficiency in the target care facility with the sleep efficiency in another care facility, to the terminal device 200E. This can present the quality of sleep to the manager. In the example of FIG. 29A, it is understood that the sleep efficiency in the own facility is higher than that in another facility, and the sleep quality of the residents is in a high state. For example, in a case where the sleep efficiency in the own facility is low, it is possible to prompt the manager to perform a response related to the sleep improvement.

FIG. 29B is a diagram in which an emotion analysis result of care receivers who occupy a care facility is displayed. For example, the processing unit 110 of the server system 100 may perform an emotion analysis to each of a plurality of care receivers based on an imaged image from the imaging device 440. For example, the server system 100 obtains a rate that each of the abovementioned plurality of emotions appears.

For example, the server system 100 may obtain an average value of the emotion rate for every care facility. The server system 100 presents information comparing an average emotion rate in a target care facility with an average emotion rate in another care facility, to the terminal device 200. This can present the emotions that the residents feel to the manager. In the example of FIG. 29B, it is understood that the rate of “HAPPINESS” as a positive emotion is higher in the own facility than in another facility, and the rates of “ANTIPATHY”, “SURPRISE”, and the like as negative emotions are lower, so that the state where no abnormalities is present in the emotion of the residents is understood. For example, in a case where the rate of the negative emotion is high in the own facility, it is possible to prompt the manger to improve the communication between the care receivers and the care givers, the living environment, and the like.

The terminal device 200E may display a result of a work evaluation related to a care giver who belongs to a target care facility. FIG. 30 is a diagram illustrating a work evaluation based on timing of an input operation. FIG. 30 is a diagram illustrating a time change in the respiratory rate of a given care receiver, and the longitudinal axis represents the respiratory rate per one minute and the horizontal axis represents time. Similar to the abovementioned example, the right side represents past in terms of time.

In the example illustrated in FIG. 30, while the respiratory rate is stable in a low state before timing Tr (at the right side in the graph), the respiratory rate increases at and after the timing Tr. It can be considered that the condition of the care receiver has suddenly changed at the timing Tr. If a care giver monitors the measurement result of a target care receiver all the time, the care giver may be able to find an abnormality at timing sufficiently close to the timing Tr. However, in the care facility and the like, one care giver is normally in charge of a plurality of care receivers, and when irregular events such as fecal leakage, wandering, a fall, and the like occur, the care giver needs to respond to the event with priority. In other words, there is a possibility that the timing when the care giver notices the abnormality varies depending on a situation and the like in which the care giver engages in care assistance work. For example, the care giver A finds the abnormality at timing Ta with a relatively short elapsed time from the timing Tr, and executes the input operation of selecting a response content. On the other hand, the care giver B cannot easily find the abnormality, and executes the input operation of selecting a response content at timing Tb with a relatively long elapsed time from the timing Tr. In this case, the probability that there are a shortage of staffs and the excessive work amount is high in a work environment of the care giver B. In other words, it is possible to use the elapsed time from the occurrence of the abnormality to the input operation for an evaluation of the work environment in the care facility and the like.

For example, the processing unit 110 may acquire abnormality occurrence timing when an abnormality has occurred in a user (care receiver, patient) based on a change in the body movement information. For example, similar to the examples mentioned above using FIGS. 23A to 24C, the processing unit 110 determines timing when the difference between the measurement results and the rate exceed predetermined thresholds as abnormality occurrence timing. In the example of FIG. 30, the abnormality occurrence timing corresponds to the timing Tr.

The processing unit 110 may then make an evaluation related to work by a second user who takes care of the abovementioned user, based on at least one of the comparison processing between abnormality occurrence timing and execution timing of the input operation in the first terminal device, and the comparison processing between abnormality occurrence timing and execution timing of the second input operation in the second terminal device. The second user herein is, for example, a care giver, but is not prevented from including a health care worker. The first terminal device is, for example, the terminal device 200A of the skilled care giver, and the second terminal device is, for example, the terminal device 200B of the unskilled care giver. In this way, by using the timing of the input operation for determining a response content, it is possible to evaluate the work environment of the care giver. The evaluation herein may be for the skilled care giver, may be for the unskilled care giver, or may be for the both. In medical facilities such as a hospital, by the similar method, an evaluation of a work environment of health care workers may be made.

For example, the processing unit 110 may perform processing of outputting an evaluation result based on a care giver who belongs to a first facility and an evaluation result based on a care giver who belongs to a second facility in a comparison possible mode. For example, the processing unit 110 calculates a difference value of an input operation by each of a plurality of care givers who belong to a given facility relative to the abnormality occurrence timing, and obtains a response time in the target facility based on an average value and the like of the difference values. When the average value is calculated herein, a weighted average that makes the degree of priority of either one of the skilled care giver and the unskilled care giver higher may be used. Although an example in which the time itself is displayed is indicated hereinafter, the processing unit 110 may perform processing of calculating an evaluation value so as to become larger as the abnormality occurrence timing and the input operation are closer to each other, and presenting the evaluation value.

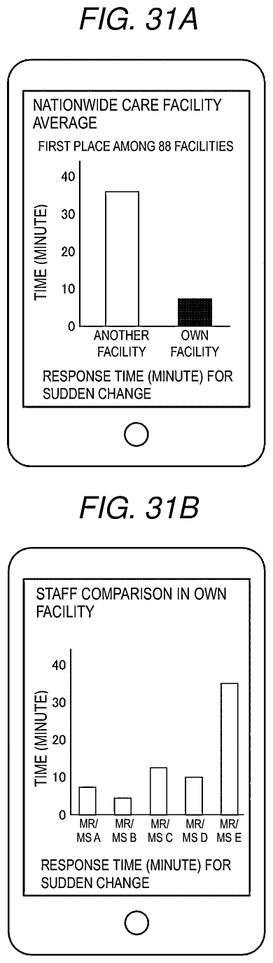

FIG. 31A illustrates an example of a screen on which a result of a work evaluation in a target facility is displayed, and an example of a screen that is displayed in the display 240 of the terminal device 200E of a manager, for example. The longitudinal axis in FIG. 31A represents a difference average value between the abnormality occurrence timing and the execution timing of the input operation. In the example of FIG. 31A, average values in the facility (own facility) of the manager and in the other plurality of facilities are displayed. In the example of FIG. 31A, the average value in the own facility is relatively low, so that it is understood that the work content has no abnormalities. On the other hand, in a case where the average value in the own facility is high, it is possible to prompt the manager to rethink the staff assignment and the work amount.

The processing unit 110 may perform processing of outputting an evaluation result related to each of a plurality of care givers who belong to a given facility in a comparison possible mode. For example, the processing unit 110 may calculate a difference value of an input operation by each of a plurality of care givers who belong to a given facility relative to the abnormality occurrence timing, and calculate an average value for every care giver.

FIG. 31B illustrates an example of a screen on which a result of a work evaluation for every care giver in a facility is displayed, and an example of a screen that is displayed in the display 240 of the terminal device 200E of a manager, for example. The longitudinal axis in FIG. 31B represents a value for every care giver between the abnormality occurrence timing and the execution timing of the input operation. In the example of FIG. 31B, an average value for each of five care givers until the execution of the input operation is displayed. The example of FIG. 31B indicates that as for care givers (Mr/Ms A, Mr/Ms B, Mr/Ms C, and Mr/Ms D) with the relatively short time, the staff assignment and the work amount are appropriate when the care givers work. On the other hand, as for a care giver (Mr/Ms E) with the relatively long time, the probability that the number of staffs is not enough when the care giver works or the work amount taken charge by the care giver is excessive is high. By displaying the screen illustrated in FIG. 31B, it is possible to prompt the manager to rethink the staff assignment and the work amount.

### 2.3 Data Administrator

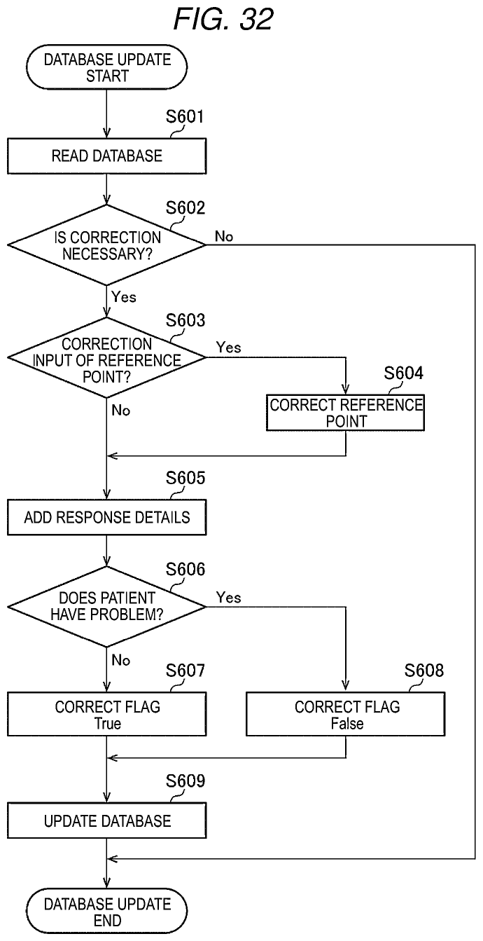

As illustrated in FIG. 25, a data administrator performs management and an update of a database. FIG. 32 is a flowchart illustrating update processing of the database by the administrator. Firstly, at Step S601, the terminal device 200F of the data administrator reads data to be managed from a database of the server system 100. In a case where the data administrator is a staff in a care facility, data to be read may be limited to information related to the target care facility. In a case where the data administrator is a staff in a medical facility, similarly, information related to the target medical facility is read. In a case where the data administrator is a service provider, at Step S601, information related to a plurality of facilities may be read.

At Step S602, the terminal device 200F determines whether the correction of data is necessary. For example, the terminal device 200F may make an inquiry about the necessity or unnecessity of the correction to the data administrator, and make the determination at Step S602 based on an operation input by the data administrator. If the correction is unnecessary (Step S602: No), the update processing of the database illustrated in FIG. 32 ends.

For example, the data administrator may perform processing of correcting a reference point based on execution timing of the input operation. The correction target herein is data of a skilled care giver to be referred as accumulated data, and the terminal device 200F may receive a correction operation of the first reference point. Data of an unskilled care giver may be an update target, and the terminal device 200F may receive a correction operation of the second reference point.

For example, at Step S603, the terminal device 200F determines whether the correction operation of a reference point has received. If the terminal device 200F has received the correction operation of the reference point (Step S603: Yes), at Step S604, the terminal device 200F transmits an operation content to the server system 100. If the terminal device 200F does not receive the correction operation of the reference point (Step S603: No), the processing at Step S604 is omitted.

It can be considered that a skilled care giver or a skilled health care worker only inputs rough responses such as “VISIT” and “ONLINE CONSULTAITON”, and determines detailed responses such as “DIAPER CHANGING” and “SHEET CHANGING” in the visit or the online consultation. In this case, the operation input by the skilled care giver and the like when finding the abnormality does not include information on the detailed responses. The terminal device 200F may receive an input operation of detail information supplementary to the response content at Step S605. The detail information herein is, as mentioned above, information for specifying the executed specific response content. At Step S605, the terminal device 200F transmits the detail information to the server system 100.

The processing unit 110 may label data based on a state thereafter of the care receiver to which the response was performed. For example, if it has determined that as a result of a response indicated by the response content, the state of a user (care receiver, patient) has become worse, the processing unit 110 may perform processing of excluding the first body movement information associated with the response content from a calculation target of similarity information with the second body movement information.

For example, at Step S606, the terminal device 200F performs processing of making an inquiry about whether a problem is present in a state of the care receiver after the response to the data administrator. For example, if the state of the care receiver has become worse after the response, the data administrator performs an input operation of the fact. The state worsening herein includes death, hospitalization, worsening of the disease condition, and the like of the care receiver.

If the terminal device 200F has received the input operation of no abnormalities (Step S606: No), at Step S607, the terminal device 200F performs processing of assigning a ground truth label with a value set to TRUE to data associated with a measurement result related to the target care receiver and the response content. Specifically the terminal device 200F requests that the server system 100 sets a ground truth flag included in the target data to TRUE.

If the terminal device 200F has received the input operation that a problem is present (Step S606: Yes), at Step S608, the terminal device 200F performs processing of assigning a ground truth label with a value set to FALSE to data associated with a measurement result related to the target care receiver and the response content. Specifically the terminal device 200F requests that the server system 100 sets a ground truth flag included in the target data to FALSE.

After the processing at Step S607 or S608, the processing unit 110 of the server system 100 performs processing of updating the target data in accordance with the request from the terminal device 200F. For example, the processing unit 110 performs the processing instructed by the data administrator, among processing of correcting the position of the reference point, processing of adding detail information on the response, and processing of setting a value of the ground truth flag. The processing of setting the ground truth flag to TRUE may be replaced with processing of performing labeling with success data. The processing of setting the ground truth flag to FALSE may be replaced with processing of performing labeling with failure data. For example, the server system 100 performs processing of excluding data with the ground truth flag set to FALSE in the reading processing of the database at Step S401 in FIG. 14 and the like. In this way, data having a possibility that the response content has not been appropriate can be excluded from the similarity calculation processing, so that the improvement in the processing accuracy becomes possible.

In a case where the update processing of the database by the data administrator is possible as the above, the number of data to which labeling (assigning a ground truth flag) has been made can be increased, so that as the processing of obtaining similar information, machine learning may be used. For example, the processing unit 110 may perform the processing of creating a learned model that uses body movement information as an input and a suitable response content as an output. As an input, environment information, an attribute, emotion information, an electronic medical record, and the like may be used. The output may be, for example, the reliability (certainty factor) that, for a plurality of response contents, the response content is appropriate.

The machine learning herein may be learning that uses Neural Network (hereinafter, described as NN), and in the narrow sense, may be learning that uses Deep Learning in which multilayer NN is used. For example, NN in the embodiment may be Recurrent Neural Network (RNN). RNN is NN in which an input at a given time point has an influence on an output at and after the given time point, and is preferable for processing of time series data. For example, NN may be Long Short Term Memory (LSTM). Machine learning is not limited to NN, but other methods such as support vector machine (SVM) and a k-means method may be used, or a method obtained by developing these methods may be used.

For example, the processing unit 110 input, in the data accumulated by the accumulation processing in FIG. 7, first body movement information as learning input data into NN, and performs forward calculation based on the weight at the time point, thereby obtaining an output. The processing unit 110 then obtains an evaluation function based on the output, and the response content that is learning ground truth data, and updates the weight based on the evaluation function. A publicly known error back-propagation method or the like is widely applicable to the update processing of the weight. For example, the processing unit 110 may obtain a learned model by using data with a ground truth flag set to TRUE as positive learning data and data with a ground truth flag set to FALSE as negative learning data.

In the use processing of the accumulated data, the processing unit 110 obtains the reliability that each response content is appropriate by inputting second body movement information into the learned model. In this case, a value of reliability the corresponds to the abovementioned similarity. As the display processing of similar information, the processing unit 110 may perform processing of causing the terminal device 200 to present the value of the reliability in association with the response content.

The example in which machine learning is performed using a result of labeling by the data administrator has been described herein, but the embodiment is not limited thereto. For example, the processing unit 110 may set a ground truth flag based on the degree of stability of the heart rate and the degree of stability of the respiratory rate of the care receiver after the execution of the response. For example, the ground truth flag is set to TRUE in a case where the variation of the heart rate or the respiratory rate is equal to or less than a predetermined threshold, and the ground truth flag is set to FALSE in a case where the variation is larger than the predetermined threshold. In this way, the ground truth flag is assigned even in a case of no update by the data administrator, so that the calculation of a similarity by machine learning becomes possible.

In addition, if the response content by the skilled worker is assumed to be reliable, setting a ground truth flag is not necessary in machine learning. For example, in the embodiment, machine learning in which all the data acquired due to the operation by the skilled worker is set as positive learning data may be performed.

### 2.4 Family

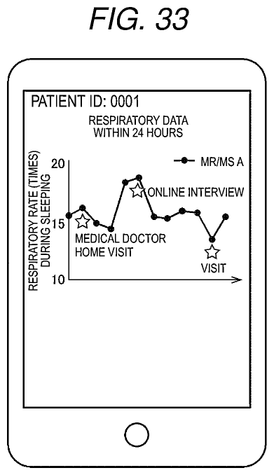

FIG. 33 illustrates an example of a screen to be displayed in the display 240 of the terminal device 200G that family of a care receiver uses. As illustrated in FIG. 33, in addition to a measurement result related to the care receiver, a response content executed by a care giver and a response content executed by a health care worker may be displayed in the terminal device 200G. In this way, it is possible to cause the family to grasp what kind of care the care receiver receives by experts in a care assistance facility, a hospital, a care receiver residence, and the like.

Although the present embodiment has been described in detail as described above, it will be readily understood by those skilled in the art that many modifications can be made without departing from the novel matters and effects of the present embodiment. Therefore, all such the modifications are intended to fall within the scope of the present disclosure. For example, a term described at least once together with a different term having a broader meaning or the same meaning in the specification or the drawings can be replaced with the different term in any part of the specification or the drawings. Moreover, all the combinations of the present embodiments and the modifications fall within the scope of the present disclosure. Further, the configuration and operation of the information processing apparatus, the information processing system, the server system, the terminal device, the measuring device, and the like are not limited to those described in the present embodiment, and various modifications can be made.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An information processing apparatus comprising:
an acquisition unit configured to acquire, based on an output from a detection device that can detect a body movement of a user who receives care assistance, body movement information including information related to a sleep state of the user;
a storing unit configured to store information in which a response content performed by a skilled worker to the user has been associated with first body movement information serving as the time series body movement information; and
a processing unit configured to perform processing of presenting, when having acquired a request including second body movement information serving as the time series body movement information, similar information including a similarity between the second body movement information and the first body movement information, and the response content associated with the first body movement information.

2. The information processing apparatus according to claim 1, wherein
the user includes a user who receives at-home care, and
the response content includes necessity or unnecessity of a visit to a residence of the user.

3. The information processing apparatus according to claim 1, wherein
the acquisition unit is configured to:
acquire, when an input operation to determine the response content has been performed in a first terminal device that the skilled worker uses, information in which the first body movement information and the response content have been associated with each other.

4. The information processing apparatus according to claim 3, wherein
the processing unit is configured to:
determine a first reference point based on execution timing of the input operation in the first terminal device, and causes the storing unit to store the body movement information in a period to be determined based on the first reference point, as the first body movement information.

5. The information processing apparatus according to claim 4, wherein
the processing unit is configured to:
determine, when the processing unit has acquired the request based on a second input operation in a second terminal device that an unskilled worker uses, a second reference point based on execution timing of the second input operation, and determines the body movement information in a period determined based on the second reference point, as the second body movement information.

6. The information processing apparatus according to claim 5, wherein
the acquisition unit is configured to:
acquire, from an environment detection device disposed in a surrounding of the user, environment information indicating an environment in the surrounding, and
the processing unit is configured to:
perform processing of correcting the first reference point and the second reference point based on a changing amount of the environment information per unit time.

7. The information processing apparatus according to any one of claim 1, wherein
the processing unit is configured to:
perform processing of obtaining first similarity information based on the first body movement information and the second body movement information in a period having a first length, and processing of obtaining second similarity information based on the first body movement information and the second body movement information in a period having a second length different from the first length, and
determine a degree of priority of the first similarity information and the second similarity information in accordance with the response content associated with the first body movement information.

8. The information processing apparatus according to any one of claim 1, wherein
the acquisition unit is configured to:
acquire, based on image information obtained by imaging the user, emotion information indicating an emotion of the user, and
the processing unit is configured to:
estimate an emotion change of the user resulting from performing a response indicated by the response content, based on the emotion information associated with the first body movement information, and performs labeling on the first body movement information based on an estimation result.

9. An information processing system comprising:
a detection device configured to detect a body movement of a user who receives care assistance;
a server system configured to acquire body movement information including information related to a sleep state of the user, based on an output from the detection device, and stores information in which a response content performed by a skilled worker to the user has been associated with first body movement information serving as the time series body movement information; and
a terminal device configured to transmit a request including second body movement information serving as the time series body movement information to the server system, wherein
the server system is configured to:
perform processing of presenting, when having acquired the request from the terminal device, similar information including a similarity between the second body movement information and the first body movement information, and the response content associated with the first body movement information.

10. An information processing method comprising:
acquiring, based on an output from a detection device that can detect a body movement of a user who receives care assistance, body movement information including information related to a sleep state of the user;
storing information in which a response content performed by a skilled worker to the user has been associated with first body movement information serving as the time series body movement information; and
performing processing of presenting, when the request including second body movement information serving as the time series body movement information has been acquired, similar information including a similarity between the second body movement information and the first body movement information, and the response content associated with the first body movement information.
